(19) United States
(12) Patent Application Publication
(10) Pub. No.: US 2023/0080665 A1
(43) Pub. Date: Mar. 16, 2023

(54) DEVICE, APPARATUS AND A METHOD FOR MINIMISING THE DISPERSAL OF PATHOGENS INTO THE ENVIRONMENT DURING INSUFFLATING OF A CAVITY IN A SUBJECT

(71) Applicant: PALLIARE LIMITED, Galway (IE)

(72) Inventors: John O'DEA, Galway (IE); Sarah KEANE, Limerick (IE); Daragh Michael MANNING, Dublin (IE); Martin James Hamilton BRUGGEMANN, Laois (IE)

(73) Assignee: PALLIARE LIMITED, Galway (IE)

(21) Appl. No.: 17/794,423

(22) PCT Filed: Mar. 26, 2021
(86) PCT No.: PCT/IE2021/000006

§ 371 Date: Jul. 21, 2022

(30) Foreign Application Priority Data
Mar. 26, 2020 (IE) S2020/0050

(51) Int. Cl. A61M 13/00; A61B 17/34

(52) U.S. Cl. CPC A61M 13/003; A61B 17/3423; A61B 2017/345; A61B 2017/3492; A61B 2217/005; A61B 2017/3419

(57) ABSTRACT

A collection device (1) for minimising dispersal of pathogens into the environment during insufflating of the peritoneal cavity (12) of a subject (3), and which are entrained in insufflating gases and other gases escaping through an incision (8) in the abdominal wall (9) of the subject (3), through which a trocar is entered into the peritoneal cavity (12) comprises a patch (5) supported on a support framework (30). The patch (5) terminates in an outer peripheral portion (20) with a pressure sensitive adhesive (21) coated thereon for sealably securing the outer peripheral portion (20) to the abdominal wall (9). A central access opening (24) formed in the patch (5) tightly and sealably engages the trocar (10). With the trocar (10) extending through the access opening (24) of the patch (5) and with the patch (5) bonded by the pressure sensitive adhesive (21) to the abdominal wall (9) of the subject, the patch (5) defines with the trocar (10) and the abdominal wall (9) of the subject an annular collection chamber (17) for collecting insufflating and other gases leaking through the opening (8) in the subject. An outlet port (25) extending from the patch (5) and communicating with the collection chamber (17) is connected through a filter (29) to a vacuum system (27) for drawing gases from the collection chamber (17) where they are filtered in the filter (29) for removing pathogens therefrom, and clean filtered gases are dispersed into the atmosphere from the vacuum system (27). Other access devices and apparatus for collecting and filtering insufflating gases and other gases leaking through an incision formed in the body of a subject or through other body orifices are also disclosed.

The invention relates to a collection device for minimising the dispersal of pathogens into the environment during insufflating of a cavity in a subject, in which a minimal invasive investigative or surgical procedure is being carried out. The invention also relates to an access device, for example, a mouth entry device, a transanal entry device or a trocar for minimising the dispersal of pathogens into the environment during insufflating of a cavity in a subject, in which a minimal invasive investigative or surgical procedure is being carried out. The invention also relates to apparatus for minimising the dispersal of pathogens into the environment during insufflating of a cavity in a subject, in which a minimally invasive investigative or surgical procedure is being carried out, and the invention also relates to a method for minimising the dispersal of pathogens into the environment during insufflating of a cavity in a subject, in which a minimal invasive investigative or surgical procedure is being carried out.

Minimal invasive investigative and surgical procedures, for example, laparoscopic procedures, in general, are carried out by gaining access to a cavity, in which an organ or vessel the subject of the procedure is located, through a trocar, an endoscope or a colonoscope. The cavity is insufflated by a suitable insufflating gas delivered into the cavity through the trocar, endoscope, colonoscope, or through an insufflating tube through the trocar or through another trocar. In the case of a trocar, the trocar or trocars are entered into the cavity through a small incision formed in the body, and surgical or other instruments are passed into the cavity through the one or more trocars. In the case of an endoscope, the endoscope may be passed into the cavity through a body orifice, such as the mouth, nose or the anus of a subject. In the case of a colonoscope, the colonoscope is passed into the cavity through a transanal entry device. However insufflating gas can leak from the relevant cavity through a trocar, an endoscope or a colonoscope, and also along the outside of the trocar, the endoscope or colonoscope through the incision or the body orifice as the case may be.

Such minimal invasive procedures have many advantages, A principal advantage of such procedures is that due to the small incisions required to be made in the subject to carry out the laparoscopic investigation or surgical procedure, recovery times from such investigative and surgical procedures are significantly shorter than corresponding recovery times from open surgical and investigative procedures. However, it has been found that while such minimal invasive investigative and surgical procedures have many advantages, they suffer from one particularly serious disadvantage, in that insufflating gases leak from the cavity being insufflated both through an instrument bore in the trocar and through the incision along the outer side of the trocar. These insufflating gases may carry pathogens, such as bacteria, infections and viruses, as well as other undesirable particulate matter which may reside in the cavity being insufflated. Additionally, smoke resulting from cauterising a vessel or the like during a procedure being carried out in the cavity being insufflated may also leak from the cavity into the environment.

Such pathogens can have serious detrimental effects on surgeons and other personnel participating in such minimal invasive surgical and investigative procedures. It is known, for example, pathogens can exist in the peritoneal cavity, and recently, it has been found that the corona virus known as COVID-19 can inhabit the peritoneal cavity. Thus, during, for example, laparoscopic surgery on or investigation of an organ or vessel in the peritoneal cavity, such pathogens, as for example, the corona virus COVID-19 may become entrained in the insufflating gases leaking from the cavity. This can in turn result in a surgeon or surgeons and other health care personnel becoming infected by such pathogens, including, the corona virus COVID-19 with consequential detrimental and in some cases fatal outcomes. This problem has become particularly serious, particularly with the onset of the corona virus COVID-19, to the extent that surgeons are now contemplating reverting to open surgery. The leaking of smoke and other particulate matter into the environment is also undesirable.

These problems are equally prevalent during procedures which are carried out in an insufflated cavity accessed orally by an endoscope or anally by an endoscope or a colonoscope. In that when an endoscope is entered orally or anally through a mouth entry device or a transanal entry device, insufflating gases may leak through an instrument bore extending through the mouth or transanal entry device and through the mouth or anus along the outer side of the mouth or transanal entry device. Such leaking insufflating gases may also carry pathogens which may reside in the subject. A similar problem arises when a colonoscope is entered into an insufflated cavity through a transanal entry device.

There is therefore a need for a device and/or an apparatus for minimising the dispersal of pathogens and other matter into the environment during insufflating of a cavity in a subject, and there is also a need for a method for minimising the dispersal of pathogens and other matter into the environment during insufflating of a cavity in a subject.

The present invention is directed towards providing such a device, as well as such an apparatus and a method.

According to the invention there is provided a collection device for minimising the dispersal of pathogens into the environment during insufflating of a lumen, vessel or cavity in a subject, the device comprising a patch terminating at an outer peripheral edge in a flexible peripheral membrane extending around and from the peripheral edge of the patch, a securing means for sealably securing the flexible peripheral membrane to a subject spaced apart around an opening through which an access device enters the subject, the patch having an access opening extending therethrough spaced apart inwardly from the outer peripheral edge thereof for accommodating the access device therethrough to the opening in the subject, the patch being adapted so that when secured to the subject by the securing means with the access device passing through the access opening in the patch and passing through the opening in the subject, the patch defines with the subject and the access device a collection chamber for collecting insufflating gas leaking through the opening in the subject around the external surface of the access device, and an outlet port is provided extending from the patch communicating with the collection chamber, the outlet port being adapted for coupling to a vacuum generating system for drawing gas from the collection chamber.

Preferably, the patch is adapted to define an annular collection chamber extending around the access device when secured to the subject by the securing means. Advantageously, the patch comprises an inner portion adjacent and extending around the access opening adapted to tightly engage the access device. Preferably, the inner portion of the patch adjacent and extending around the access opening is adapted to sealably engage the access device.

In one embodiment of the invention the inner portion of the patch adjacent and extending around the access opening comprises a resilient material, and preferably, the inner portion of the patch adjacent and extending around the access opening comprises a flexible material, and ideally, the inner portion of the patch adjacent and extending around the access opening comprises an elastic material.

In another embodiment of the invention the patch comprises an intermediate portion located between the peripheral membrane and the inner portion of the patch extending around the inner portion, the intermediate portion of the patch comprising a flexible material.

In another embodiment of the invention at least the intermediate portion of the patch comprises a semi-rigid material.

In a further embodiment of the invention at least the intermediate portion of the patch comprises a substantially self-supporting material.

Alternatively, the intermediate portion of the patch comprises a non-self-supporting material.

Preferably, the patch comprises a material impermeable to insufflating gases.

Advantageously, the patch is integrally formed from a single sheet of material.

In one embodiment of the invention a support element is provided for supporting the patch to define with the access device and the subject the collection chamber.

Preferably, the support element is adapted for locating between the patch and the subject, and advantageously, the support element is adapted to extend around the patch spaced apart from the access opening.

Preferably, the support element comprises a support framework, and advantageously, the support framework comprises a lower peripheral frame for locating adjacent the subject, and an upper peripheral frame spaced apart above the lower peripheral frame, the upper peripheral frame being joined to the lower peripheral frame by at least one strut extending from the upper peripheral frame to the lower peripheral frame.

Preferably, the upper peripheral frame extends around but spaced apart outwardly from the access opening, and advantageously, the upper peripheral frame is spaced apart outwardly from the access opening to permit flexing of the inner portion of the patch. Preferably, the lower peripheral frame extends around adjacent but spaced apart inwardly from the outer peripheral edge of the patch.

Preferably, the upper peripheral frame comprises an upper circular ring member, and advantageously, the lower peripheral frame comprises a lower circular ring member.

In one embodiment of the invention the diameter of the lower peripheral frame is greater than the diameter of the upper peripheral frame.

In another embodiment of the invention the securing means comprises an adhesive coated onto the peripheral membrane.

Preferably, the peripheral edge in which the patch terminates defines a circle.

Preferably, the access opening is of circular shape.

In one embodiment of the invention the access opening is adapted to accommodate a trocar therethrough.

In another embodiment of the invention the access opening is adapted to accommodate an endoscope.

In a further embodiment of the invention the access opening is adapted to accommodate a colonoscope.

In one embodiment of the invention a filter is provided for filtering gases drawn from the collection chamber.

In another embodiment of the invention the filter is adapted for coupling to the outlet port.

Preferably, the filter is adapted to filter out one or more of pathogens, bacteria, infections and viruses from the gases passing therethrough.

In one embodiment of the invention the filter is adapted to filter out particles of size greater than 0.1 microns entrained in the gases passing therethrough, and preferably, the filter is adapted to filter out particles of size greater than 0.05 microns entrained in the gases passing therethrough, and advantageously, the filter is adapted to filter out particles of size greater than 0.04 microns entrained in the gases passing therethrough.

The invention also provides an access device for accommodating an instrument to a lumen, vessel or cavity in a subject, and the collection device according to the invention located on the access device with the access device extending through the access opening in the patch and sealably engaged in the access opening.

The invention also provides a trocar comprising the collection device according to the invention located on the trocar with the trocar extending through the access opening in the patch and sealably engaged in the access opening.

The invention also provides a method for minimising dispersal of pathogens into the environment during insufflating of a lumen, vessel or cavity of a subject, the method comprising:
providing a patch terminating in an outer peripheral edge and having an access opening extending therethrough spaced apart inwardly from the peripheral edge thereof,
placing the patch topically on an area of the skin of the subject containing an opening through which an access device enters the subject,
sealably securing the patch to the skin of the subject adjacent the peripheral edge thereof spaced apart from and extending around the opening in the subject, entering the access instrument through the access opening to the opening in the subject with a portion of the patch adjacent the access opening sealably engaging the access device with the patch defining with the access device and the skin of the subject a collection chamber for collecting insufflating gas leaking from the opening in the subject along the outer surface of the access device, and
applying a vacuum to an outlet port from the patch to draw the insufflating gases from the collection chamber.

In one embodiment of the invention the patch is sealably secured to the skin of the subject with the patch defining with the access device and the skin of the subject the collection chamber in the form of an annular collection chamber.

Preferably, the patch comprises an inner portion adjacent and extending around the access opening adapted to tightly engage the access device.

Preferably, the patch is secured to the skin of the subject by an adhesive.

Additionally, the invention provides an access device for accommodating an instrument through an opening in a human or animal subject, the access device being adapted to extend through the opening in the subject and having an instrument bore extending therethrough for accommodating the instrument therethrough, the access device having an inner surface defining the instrument bore, and an external outer surface, wherein the access device comprises a collection chamber located in the access device between and spaced apart from the inner and outer surfaces thereof and extending at least partly around the instrument bore, an outlet port communicating with the collection chamber and adapted for connecting to a vacuum system, and at least one first inlet port communicating with and extending from the collection chamber and terminating in one of an inner surface and an outer surface of the access device, the at least one first inlet port being adapted to draw gases into the collection chamber in response to a vacuum being applied to the outlet port.

Preferably, the collection chamber comprises an annular chamber extending around the instrument bore. Advantageously, the collection chamber extends completely around the instrument bore.

In one embodiment of the invention the at least one first inlet port terminates in the one of the inner surface and the outer surface of the access device towards a proximal end thereof.

In another embodiment of the invention the at least one first inlet port terminates in the one of the inner surface and the outer surface of the access device adjacent the proximal end thereof.

In another embodiment of the invention the at least one first inlet port terminates in the outer surface of the access device for drawing gases leaking through the opening in the subject past the outer surface of the access device.

Preferably, a plurality of the first inlet ports extend from and communicate with the collection chamber and terminate in the outer surface of the access device spaced apart circumferentially around the access device, and advantageously, the first inlet ports are equi-spaced apart circumferentially around the access device.

In another embodiment of the invention at least one second inlet port communicates with and extends from the collection chamber and terminates in the inner surface of the access device, the at least one second inlet port being adapted to draw gases into the collection chamber in response to a vacuum being applied to the outlet port.

Preferably, the at least one second inlet port communicates with the instrument bore of the access device.

In another embodiment of the invention the at least one second inlet port terminates in the inner surface of the access device towards the proximal end thereof, and preferably, the at least one second inlet port terminates in the inner surface of the access device adjacent the proximal end thereof.

Preferably, a plurality of the second inlet ports extend from and communicate with the collection chamber and terminate in the inner surface of the access device spaced apart circumferentially around the inner surface, and advantageously, the second inlet ports are equi-spaced apart circumferentially around the inner surface of the access device.

Preferably, the collection chamber is located towards the proximal end of the access device, and advantageously, the collection chamber is located adjacent the proximal end of the access device.

In one embodiment of the invention the access device terminates in one of a proximal flange and a proximal housing, the one of the proximal flange and the proximal housing defining a part of the outer surface of the access device.

In another embodiment of the invention the at least one first inlet port terminates in the outer surface of the access device adjacent but spaced apart distally from the proximal housing.

In an alternative embodiment of the invention the at least one first inlet port terminates in the part of the outer surface of the access device defined by the proximal housing.

In one embodiment of the invention the at least one first inlet port terminates in the part of the outer surface of the access device defined by the proximal housing towards the access device.

Advantageously, the at least one first inlet port terminates in the part of the outer surface of the access device defined by the proximal housing adjacent but spaced apart from the access device.

In another embodiment of the invention the at least one first inlet port terminates in the outer surface of the access device intermediate the access device and the proximal housing.

Alternatively, the at least one first inlet port terminates in the part of the outer surface of the access device defined by the proximal housing spaced apart from the access device.

In another embodiment of the invention the at least one first inlet port terminates in the outer surface of the access device adjacent but spaced apart distally from the proximal flange.

Alternatively, the at least one first inlet port terminates in the part of the outer surface of the access device defined by the proximal flange.

Preferably, the at least one first inlet port terminates in the part of the outer surface of the access device defined by the proximal flange towards the access device, and advantageously, the at least one first inlet port terminates in the part of the outer surface of the access device defined by the proximal flange adjacent but spaced apart from the access device.

Alternatively, the at least one first inlet port terminates in the outer surface of the access device intermediate the access device and the proximal flange.

In a further embodiment of the invention the at least one first inlet port terminates in the part of the outer surface of the access device defined by the proximal flange spaced apart from the access device.

Preferably, the outlet port extends from the collection chamber and terminates in the part of the outer surface of the access device formed by the one of the proximal flange and the proximal housing, and advantageously, the outlet port extends from the collection chamber and terminates in or extends through the outer surface of the access device.

Preferably, a filter is provided for filtering out one or more of pathogens, bacteria, infections and viruses from gases drawn through the outlet port, and advantageously, the filter is adapted for coupling to the outlet port.

In one embodiment of the invention the outlet port from the collection chamber is adapted for coupling to a vacuum system, and preferably, the vacuum system is adapted to generate a vacuum of pressure in the range of 150 mmHg to 300 mmHg below atmospheric pressure for applying to the outlet port from the collection chamber, and preferably, for applying to the outlet port through a filter.

In one embodiment of the invention the access device comprises a trocar.

In another embodiment of the invention the access device comprises a mouth entry device for accommodating an instrument orally into the subject.

In a further embodiment of the invention the access device comprises a transanal entry device for accommodating an instrument anally into the subject.

Further the invention provides a trocar for accommodating an instrument through an incision formed in a human or animal subject, the trocar being adapted to extend through the incision in the subject and having an instrument bore extending therethrough for accommodating the instrument therethrough, the trocar having an inner surface defining the instrument bore, and an external outer surface, wherein the trocar comprises a collection chamber located in the trocar between and spaced apart from the inner and outer surfaces thereof and extending at least partly around the instrument bore, an outlet port communicating with the collection chamber and adapted for connecting to a vacuum system, and at least one first inlet port communicating with and extending from the collection chamber and terminating in one of the inner surface and the outer surface of the trocar, and the at least one first inlet port being adapted to draw gases into the collection chamber in response to a vacuum being applied to the outlet port.

In one embodiment of the invention the collection chamber comprises an annular chamber extending around the instrument bore.

Preferably, the collection chamber extends completely around the instrument bore.

In one embodiment of the invention the at least one first inlet port terminates in the one of the inner surface and the outer surface of the trocar towards a proximal end thereof, and preferably, the at least one first inlet port terminates in the one of the inner surface and the outer surface of the trocar adjacent the proximal end thereof.

In another embodiment of the invention the at least one first inlet port terminates in the outer surface of the trocar for drawing the gases leaking through the incision in the subject past the outer surface of the trocar.

Preferably, a plurality of the first inlet ports extend from and communicate with the collection chamber and terminate in the outer surface of the trocar spaced apart circumferentially around the trocar, and advantageously, the first inlet ports are equi-spaced apart circumferentially around the trocar.

In another embodiment of the invention at least one second inlet port communicates with and extends from the collection chamber and terminates in the inner surface of the trocar, the at least one inlet port being adapted to draw gases into the collection chamber in response to a vacuum being applied to the outlet port.

Preferably, the at least one second inlet port communicates with the instrument bore of the trocar, and preferably, the at least one second inlet port terminates in the inner surface of the trocar towards the proximal end thereof, and advantageously, the at least one second inlet port terminates in the inner surface of the trocar adjacent the proximal end thereof.

Preferably, a plurality of the second inlet ports communicate with and extend from the collection chamber and terminate in the inner surface of the trocar spaced apart circumferentially around the inner surface, and advantageously, the second inlet ports are equi-spaced apart circumferentially around the inner surface of the trocar.

Preferably, the collection chamber is located towards the proximal end of the trocar, and advantageously, the collection chamber is located adjacent the proximal end of the trocar.

In one embodiment of the invention the trocar terminates in a proximal housing, the proximal housing defining a part of the outer surface of the trocar.

Preferably, the at least one first inlet port terminates in the outer surface of the trocar adjacent but spaced apart distally from the proximal housing.

Alternatively, the at least one first inlet port terminates in the part of the outer surface of the trocar forming the proximal housing.

In one embodiment of the invention the at least one first inlet port terminates in the part of the outer surface of the trocar defined by the proximal housing towards the trocar, and preferably, the at least one first inlet port terminates in the part of the outer surface of the trocar defined by the proximal housing adjacent but spaced apart from the trocar.

In another embodiment of the invention the at least one first inlet port terminates in the outer surface of the trocar intermediate the trocar and the proximal housing.

Alternatively, the at least one first inlet port terminates in the part of the outer surface of the trocar defined by the proximal housing spaced apart from the trocar.

In one embodiment of the invention the outlet port terminates in the part of the outer surface of the trocar forming the proximal housing.

Preferably, the outlet port extending from the collection chamber terminates in or extends through the outer surface of the trocar.

Preferably, a filter is provided for filtering out one or more of pathogens, bacteria, infections and viruses from gases drawn through the outlet port.

Advantageously, the filter is adapted for coupling to the outlet port.

The invention also provides a mouth entry device for accommodating an instrument through the mouth of a human or animal subject, the mouth entry device being adapted to extend through the mouth of the subject and having an instrument bore extending therethrough for accommodating the instrument therethrough, the mouth entry device having an inner surface defining the instrument bore, and an external outer surface, wherein the mouth entry device comprises a collection chamber located in the mouth entry device between and spaced apart from the inner and outer surfaces thereof and extending at least partly around the instrument bore, an outlet port communicating with the collection chamber and adapted for connecting to a vacuum system, and at least one first inlet port communicating with and extending from the collection chamber and terminating in one of the inner surface and the outer surface of the mouth entry device, the at least one first inlet port being adapted to draw gases into the collection chamber in response to a vacuum being applied to the outlet port.

Preferably, the collection chamber comprises an annular chamber extending around the instrument bore.

Advantageously, the collection chamber extends completely around the access bore.

Preferably, the at least one first inlet port terminates in the one of the inner surface and the outer surface of the mouth entry device towards a proximal end thereof.

Preferably, the at least one first inlet port terminates in the one of the inner surface and the outer surface of the mouth entry device adjacent the proximal end thereof.

In one embodiment of the invention the at least one first inlet port terminates in the outer surface of the mouth entry device for drawing gases leaking through the mouth of the subject past the outer surface of the mouth entry device.

Preferably, a plurality of first inlet ports extend from and communicate with the collection chamber and terminate in the outer surface of the mouth entry device spaced apart circumferentially around the mouth entry device, and advantageously, the first inlet ports are equi-spaced apart circumferentially around the mouth entry device.

In another embodiment of the invention at least one second inlet port communicates with and extends from the collection chamber and terminates in the inner surface of the mouth entry device, the at least one second inlet port being adapted to draw gases into the collection chamber in response to a vacuum being applied to the outlet port.

Preferably, the at least one second inlet port communicates with the instrument bore of the mouth entry device.

Advantageously, the at least one second inlet port terminates in the inner surface of the mouth entry device towards the proximal end thereof, and advantageously, the at least one second inlet port terminates in the inner surface of the mouth entry device adjacent the proximal end thereof.

Preferably, a plurality of the second inlet ports communicate with and extend from the collection chamber and terminate in the inner surface of the mouth entry device spaced apart circumferentially around the inner surface, and advantageously, the second inlet ports are equi-spaced apart circumferentially around the inner surface of the mouth entry device.

In one embodiment of the invention the outlet port extending from the collection chamber terminates in or extends through the outer surface of the mouth entry device, and preferably, the collection chamber is located towards the proximal end of the mouth entry device.

Preferably, the collection chamber is located adjacent the proximal end of the mouth entry device.

Preferably, a filter is provided for filtering out one or more of pathogens, bacteria, infections and viruses from gases drawn through the outlet port, and advantageously, the filter is adapted for coupling to the outlet port.

The invention also provides a transanal entry device for accommodating an instrument through the anus of a human or animal subject, the transanal entry device being adapted to extend through the anus in the subject and having an instrument bore extending therethrough for accommodating the instrument therethrough, the transanal entry device having an inner surface defining the instrument bore, and an external outer surface, wherein the transanal entry device comprises a collection chamber located in the transanal entry device between and spaced apart from the inner and outer surfaces thereof and extending at least partly around the instrument bore, an outlet port communicating with the collection chamber and adapted for connecting to a vacuum system, at least one first inlet port communicating with and extending from the collection chamber and terminating in one of the inner surface and the outer surface, the at least one first inlet port being adapted to draw gases into the collection chamber in response to a vacuum being applied to the outlet port.

Preferably, the collection chamber comprises an annular chamber extending around the instrument bore.

Advantageously, the collection chamber extends completely around the access bore.

Preferably, the at least one first inlet port terminates in the one of the inner surface and the outer surface of the transanal entry device towards a proximal end thereof, and advantageously, the at least one first inlet port terminates in the one of the inner surface and the outer surface of the transanal entry device adjacent the proximal end thereof.

In one embodiment of the invention the at least one first inlet port terminates in the outer surface of the transanal entry device for drawing gases leaking through the anus of the subject past the outer surface of the transanal entry device.

Preferably, a plurality of first inlet ports communicate with and extend from the collection chamber and terminate in the outer surface of the transanal entry device spaced apart circumferentially around the transanal entry device.

Advantageously, the first inlet ports are equi-spaced apart circumferentially around the transanal entry device.

In another embodiment of the invention at least one second inlet port communicates with and extends from the collection chamber and terminates in the inner surface of the transanal entry device, the at least one second inlet port being adapted to draw gases into the collection chamber in response to a vacuum being applied to the outlet port.

Preferably, the at least one second inlet port communicates with the instrument bore of the transanal entry device.

Preferably, the at least one second inlet port terminates in the inner surface of the transanal entry device towards the proximal end thereof, and advantageously, the at least one second inlet port terminates in the inner surface of the transanal entry device adjacent the proximal end thereof.

Preferably, a plurality of the second inlet ports communicate with and extend from the collection chamber and terminate in the inner surface of the transanal entry device spaced apart circumferentially around the inner surface, and advantageously, the second inlet ports are equi-spaced circumferentially around the inner surface of the transanal entry device.

Preferably, the outlet port extending from the collection chamber terminates in or extends through the outer surface of the transanal entry device.

Preferably, the collection chamber is located towards the proximal end of the transanal entry device, and advantageously, the collection chamber is located adjacent the proximal end of the transanal entry device.

Preferably, a filter is provided for filtering out one or more of pathogens, bacteria, infections and viruses from gases drawn through the outlet port, and preferably, the filter is adapted for coupling to the outlet port.

Further the invention provides a method for minimising dispersal of gases escaping from an opening in a subject through which an access device is located for accommodating an instrument into the subject, the method comprising providing the access device with an instrument bore extending therethrough for accommodating the instrument therethrough and providing the access device with an inner surface defining the instrument bore therethrough and an external outer surface, providing a collection chamber located in the access device between and spaced apart the inner and outer surfaces thereof and extending at least partly around the instrument bore providing at least one outlet port communicating with the collection chamber adapted to communicate with a vacuum system, providing at least one first inlet port communicating with and extending from the collection chamber and terminating in one of the inner surface and the outer surface of the access device, locating the access device in the opening in the subject, and applying a vacuum to the outlet port for drawing gases escaping from the subject into the collection chamber through the at least one first inlet port.

In one embodiment of the invention the access device comprises a trocar, and the trocar is located in the opening formed by an incision formed in the subject with the at least one first inlet port located externally of the incision but adjacent thereto.

In another embodiment of the invention the access device comprises a mouth entry device for accommodating an instrument orally into the subject, and the mouth entry device is located in the mouth of the subject between the upper and lower teeth of the subject with the at least one first inlet port located in or externally of the mouth of the subject but adjacent the mouth of the subject.

In a further embodiment of the invention the access device comprises a transanal entry device for accommodating an instrument anally into the subject, and the transanal entry device is located in the anus of the subject, with the at least one first inlet port located externally of the anus but adjacent thereto.

The invention also provides apparatus for minimising the dispersal of pathogens into the environment during insufflating of a cavity in a human or animal subject, the apparatus comprising a plurality of collection element connected in a daisy-chain configuration and adapted to be placed on the subject around a site where one or more access devices extend into the subject through one or more corresponding openings in the subject, each collection element having at least one outlet port adapted for coupling to a vacuum system, and at least one inlet port communicating with the outlet port and adapted to draw gases therethrough escaping from the one or more openings in the subject in response to a vacuum being applied to the outlet port.

Preferably, a securing means is provided for securing each collection element to the subject, and advantageously, the securing means comprises an adhesive for bonding the corresponding collection element to the subject.

In one embodiment of the invention each collection element defines a collection chamber, and the at least one inlet port communicates with the collection chamber.

Preferably, the outlet port of each collection element communicates with the collection chamber thereof.

In one embodiment of the invention each collection element comprises a base wall and a first side wall extending upwardly from the base wall, and the at least one inlet port of the collection element extends through the first side wall.

Preferably, each collection element comprises a top wall spaced apart from the base wall, the first side wall extending between the base wall and the top wall, and a second side wall extending between the base wall and the top wall and extending from respective opposite ends of the first side wall, the base wall, the top wall, the first side wall and the second side wall together defining the collection chamber.

Advantageously, the outlet port of each collection element extends through the second side wall.

Preferably, the base wall defines a base surface, and the securing means is located on the base surface.

Advantageously, adjacent ones of the collection elements area joined by a connecting element, and preferably, each connecting element comprises one of a chain and a tether.

Preferably, the collection elements are configured in the form of an endless daisy-chain.

In one embodiment of the invention a manifold comprising an outlet port adapted for coupling to a vacuum system is provided, the manifold comprising a plurality of inlet ports coupled to the outlet ports of the respective collection elements.

Preferably, the manifold comprises an elongated conduit, and the inlet ports thereof are spaced apart longitudinally along the conduit, and advantageously, the conduit comprises an endless conduit configured to extend around the collection elements when placed on the site of the subject.

Preferably, a filter is provided for filtering gases drawn through the outlet port of the manifold, and advantageously, the filter is adapted to filter out one or more of pathogens, bacteria, infections and viruses from gases passing therethrough.

The invention also provides a system for minimising the dispersal of pathogens into the environment during insufflating of a cavity in a human or animal subject, the system comprising the apparatus according to the invention and a vacuum system coupled to the outlet port of the manifold for applying a vacuum thereto.

Additionally, the invention provides a method for minimising the dispersal of pathogens into the environment during insufflating of a cavity in a human or animal subject, the method comprising placing the collection elements configured in the daisy-chain configuration of the apparatus according to the invention on a subject around a site where one or more access devices extend into the subject through one or more openings in the subject, the collection elements being placed on the subject with the inlet ports thereof facing inwardly towards the site, and applying a vacuum to the outlet ports of the respective collection elements for drawing gases escaping from the openings in the subject through the inlet ports of the collection elements.

Preferably, the manifold of the apparatus is placed on the subject extending around the collection elements with the inlet ports of the manifold coupled to the outlet ports of the respective collection elements.

Advantageously, a vacuum is applied to the outlet port of the manifold.

Preferably, gases drawn through the outlet port of the manifold are filtered for filtering out one or more of pathogens, bacteria, infections and viruses from the gases.

Further the invention provides a collection device for minimising the dispersal of pathogens into the environment during insufflating of a lumen, vessel or cavity in a subject, the collection device comprising a pair of arcuate members, each arcuate member extending between a first end and a second end and having a collection chamber therein, the arcuate members being hingedly coupled together adjacent the first ends thereof and being hingeable from a closed state with the second ends abutting each other and the arcuate members defining an access bore therethrough for accommodating an access device or an instrument therethrough, and an open state with the second ends spaced apart for accommodating an access device or an instrument radially into the access bore, an outlet port extending from one of the arcuate members and communicating with the collection chamber, and at least one inlet port communicating with the collection chamber and terminating in the access bore for drawing gases therein into the collection chamber in response to a vacuum being applied to the outlet port.

Preferably, the collection chambers in the respective arcuate members communicate with each other through the first ends thereof when the arcuate members are in the closed state thereof.

Advantageously, the communicating chambers in the respective arcuate members communicate with each other through the second ends thereof when the arcuate members are in the closed state thereof.

In one embodiment of the invention a fastening means is provided for securing the second ends of the arcuate members in the closed state.

Preferably, the fastening means comprises a releasable fastening means.

Advantageously, the fastening means comprises a pair of interengageable complementary formations, one of the interengageable complementary formations being located on the second end of one of the arcuate members, and the other one of the interengageable complementary formations being located on the second end of the other one of the arcuate members, and preferably, the interengageable complementary formations interengage with a releasable snap-fit action.

Preferably, each arcuate member comprises a plurality of the inlet ports spaced apart circumferentially around the access bore when the arcuate members are in the closed state.

In another embodiment of the invention a closure film is located closing the access bore at one end thereof, the closure film having a central bore extending therethrough for accommodating the access device or the instrument therethrough, and a radial slit extending from the central bore to the second ends of the arcuate members, the closure film being of an elastic material for permitting hinging of the arcuate members from the closed state to the open state.

In a further embodiment of the invention a securing means is provided for releasably securing the collection device to the skin of a subject.

Preferably, each arcuate member defines a part of a circle.

Advantageously, each arcuate member is of semi-circular shape.

The advantages of the invention are many. A particular important advantage of the invention is that the dispersal of pathogens entrained in insufflating gases and other gases being expelled from or leaking from an insufflated cavity in a subject during a minimally invasive investigative or surgical procedure is minimised. By collecting the expelled or leaking insufflating gases and other gases in the collection chamber, the gases can then be drawn from the collection chamber and filtered to remove pathogens and other particulate matter, and clean filtered gases are then safely dispersed into the environment. This is a particularly important advantage of the invention, since it minimises, if not totally avoiding any danger of the environment around a subject and other personnel involved in carrying out or assisting in the procedure being exposed to pathogens and other particulate matter, thereby minimising, if not totally avoiding, any risk to a surgeon and other such personnel being infected with such pathogens.

The advantage of the collection device comprising the patch which defines with a trocar and an adjacent part of the subject a collection chamber, is that substantially all insufflating gases and other gases leaking through the incision in the subject, through which the trocar or other access instrument enters the subject, are collected in the collection chamber, and are then drawn under vacuum from the collection chamber and filtered prior to being dispersed into the environment in order to filter out pathogens and other particulate matter from the leaked insufflating and other gases, and thereby clean filtered gases are safely dispersed into the environment.

The advantage of the access device according to the invention, whether the access device is provided as a mouth entry device, a transanal entry device or a trocar, is that insufflating gas being expelled through the instrument bore of the access device, as well as insufflating gas leaking through the incision or other body orifice, through which the access device is entered into the subject, are collected. The collected insufflating gases and other gases are then filtered for filtering out pathogens and other particulate matter, so that the clean filtered gases are then safely dispersed into the environment.

Similar advantages are derived from the apparatus according to the invention whereby a plurality of collecting elements configured in the form of a daisy-chain, whereby insufflating gas escaping through incisions in the subject, through which one or more trocars and/or other access devices are entered into a subject, are collected in the collection elements and are then filtered for removing pathogens and other particulate matter, so that clean filtered gases are dispersed safely into the environment.

The invention will be more clearly understood from the following description of some preferred embodiments thereof which are given by way of example only with reference to the accompanying drawings, in which:

FIG. 1 is a side elevational view of a collection device according to the invention for minimising the dispersal of pathogens into the environment during insufflating of a cavity in a subject,

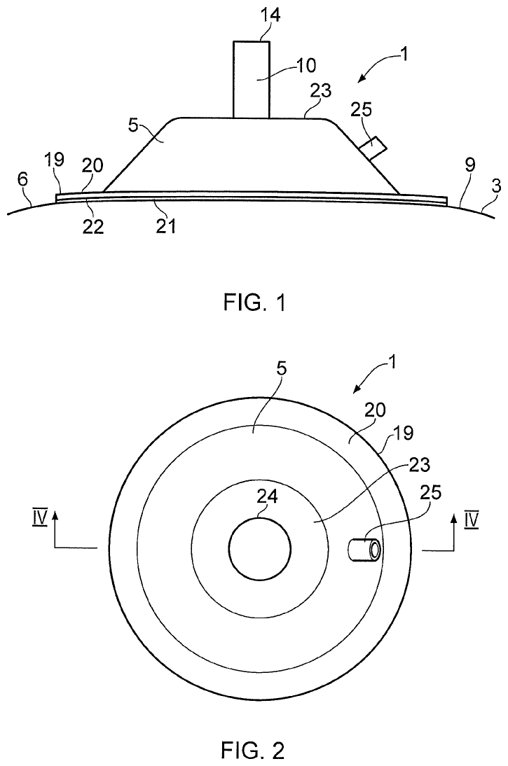

FIG. 2 is a top plan view of the collection device of FIG. 1,

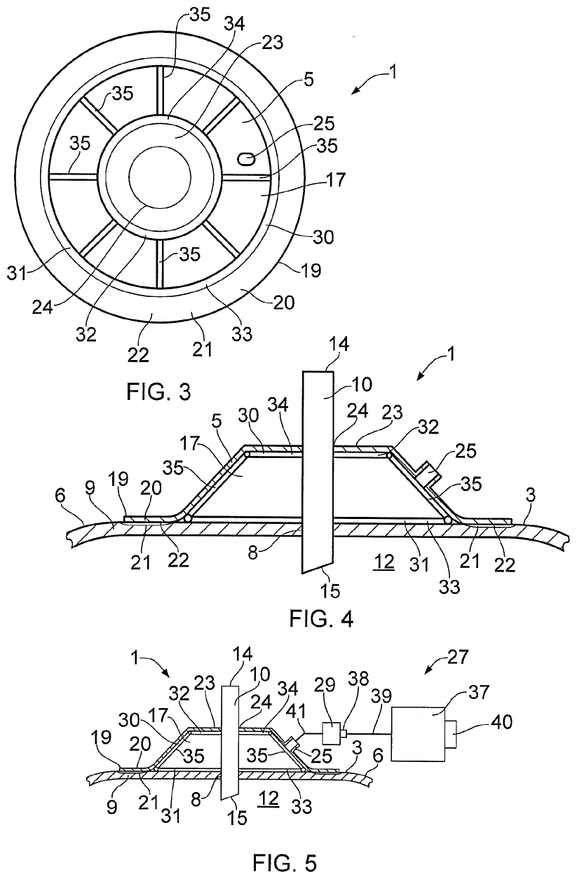

FIG. 3 is an underneath plan view of the collection device of FIG. 1,

FIG. 4 is a cross-sectional side elevational view of the collection device of FIG. 1 in use on the line IV-IV of FIG. 2,

FIG. 5 is a partly block representational view of the collection device of FIG. 2, in use,

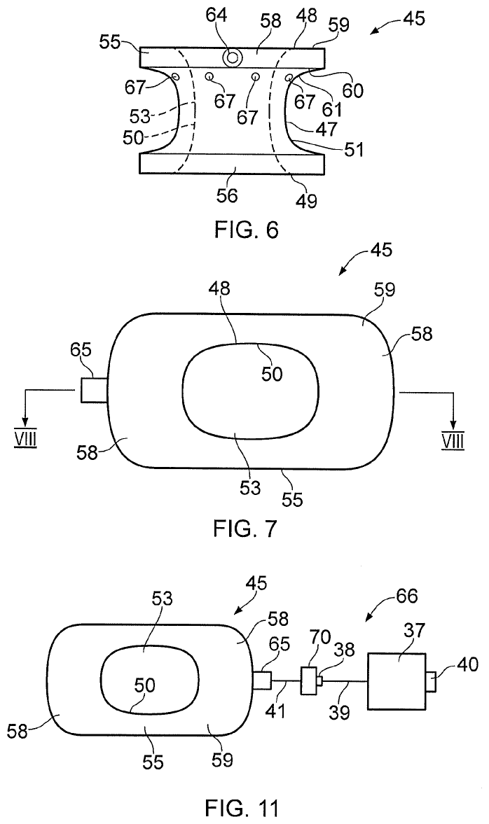

FIG. 6 is an end elevational view of an access device according to the invention for accommodating an instrument through an orifice in a subject for minimising the dispersal of pathogens into the environment during insufflating of a cavity in the subject,

FIG. 7 is a top plan view of the access device of FIG. 6,

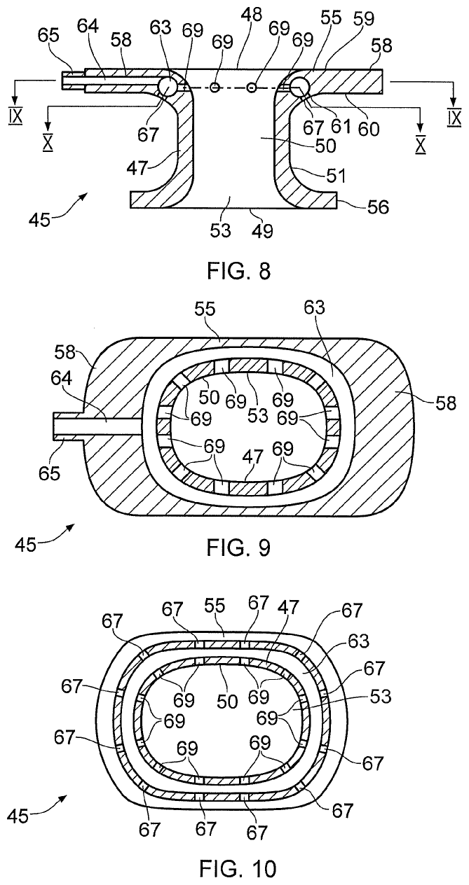

FIG. 8 is a cross-sectional side elevational view of the access device of FIG. 6 on the line VIII-VIII of FIG. 6,

FIG. 9 is a cross-sectional top plan view of the access device of FIG. 6 on the lines IX-IX of FIG. 8,

FIG. 10 is a cross-sectional top plan view of the access device of FIG. 6 on the line X-X of FIG. 8,

FIG. 11 is a partly block representational view of the access device of FIG. 6, in use,

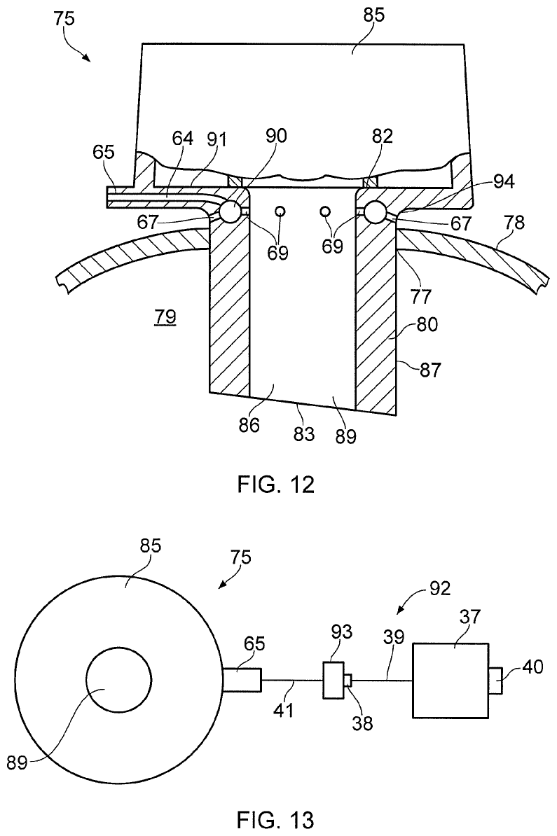

FIG. 12 is a cross-sectional side elevational view of another access device also according to the invention,

FIG. 13 is a partly block representational plan view of the access device of FIG. 12,

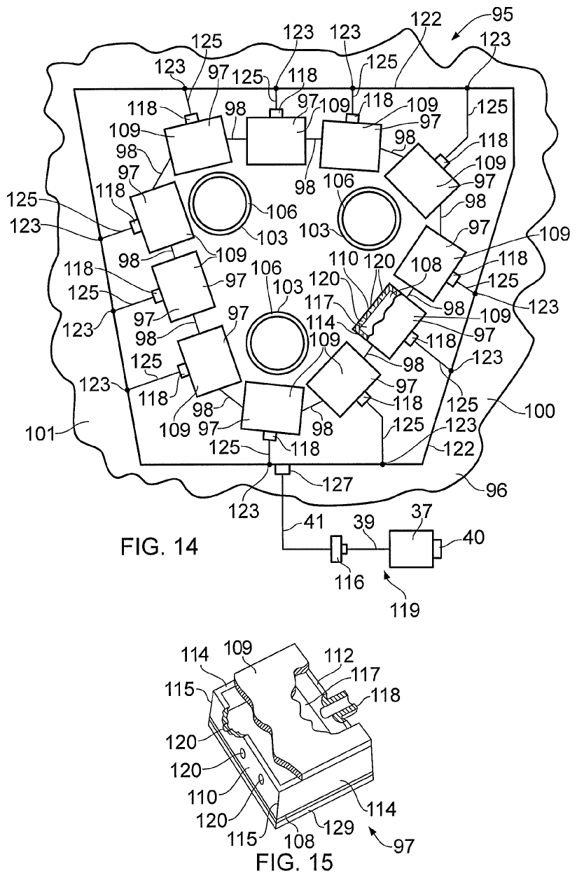

FIG. 14 is a partly block representational view of apparatus also according to the invention in use for minimising the dispersal of pathogens into the environment during the insufflating of a cavity in a subject,

FIG. 15 is an enlarged perspective view of a detail of the apparatus of FIG. 14,

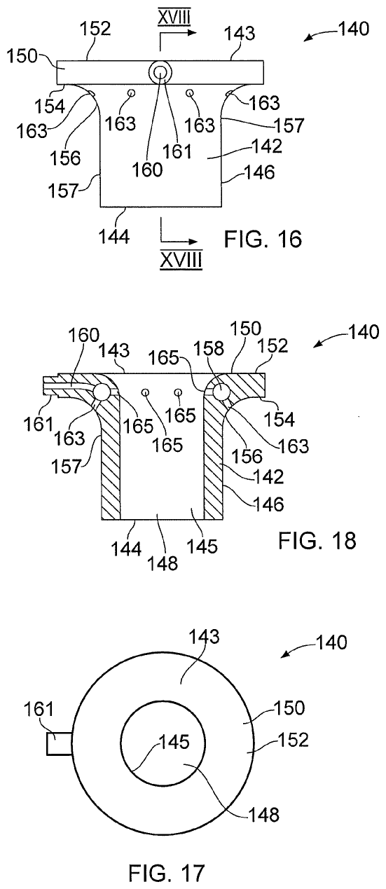

FIG. 16 is an end elevational view of another access device according to another embodiment of the invention,

FIG. 17 is a top plan view of the access device of FIG. 16,

FIG. 18 is a cross-sectional side elevational view of the access device of FIG. 16 on the line XVIII-XVIII of FIG. 16,

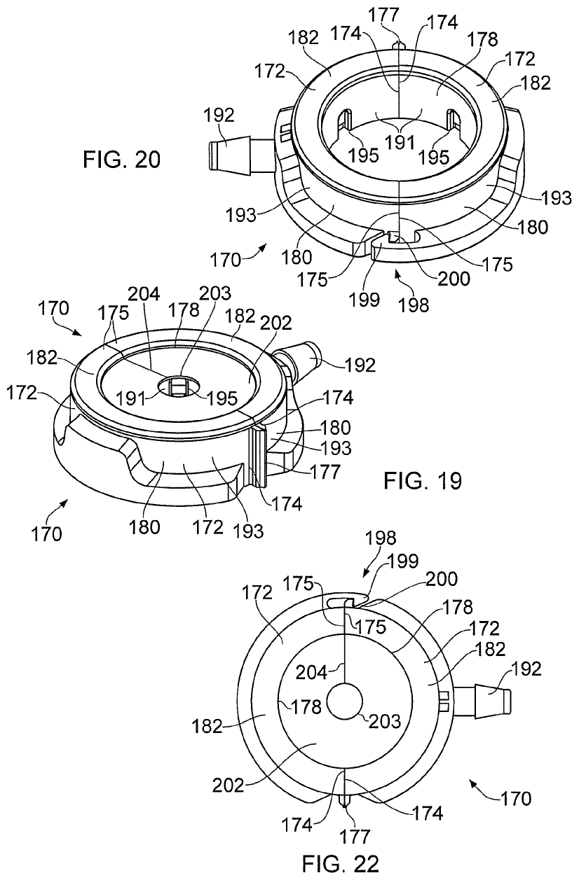

FIG. 19 is a perspective view of a collection device according to another embodiment of the invention,

FIG. 20 is another perspective view of the collection device of FIG. 19 with a part of the collection device removed,

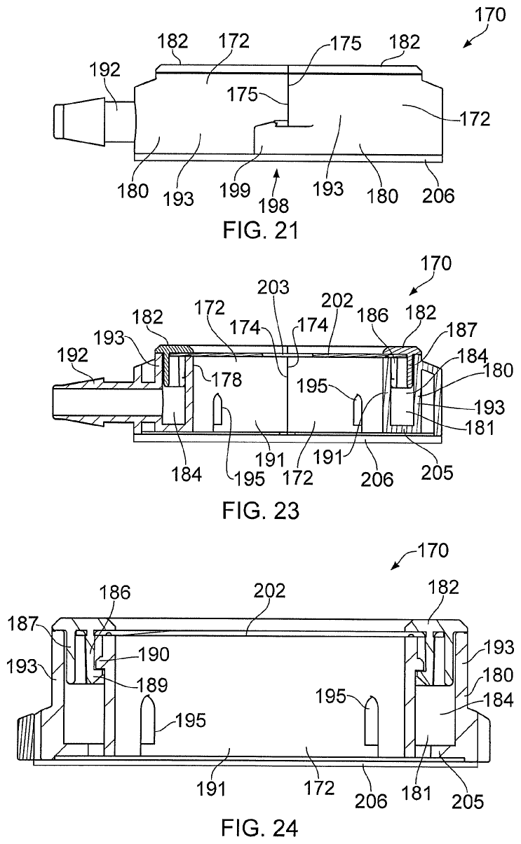

FIG. 21 is a side elevational view of the collection device of FIG. 19,

FIG. 22 is a top plan view of the collection device of FIG. 19,

FIG. 23 is a cross-sectional side elevational view of the collection device of FIG. 19 on the line XXIII-XXIII of FIG. 22,

FIG. 24 is a cross-sectional end elevational view of the collection device of FIG. 19 on the line XXIV-XXIV of FIG. 22,

FIG. 25 is an end elevational view of the collection device of FIG. 19 in a different state to that of FIG. 19,

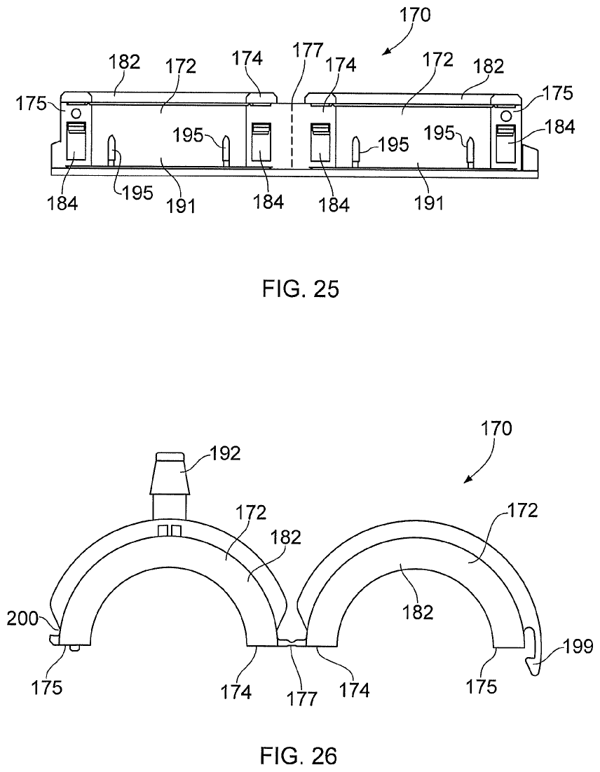

FIG. 26 is a top plan view of the collection device of FIG. 19 in the state of FIG. 25, and

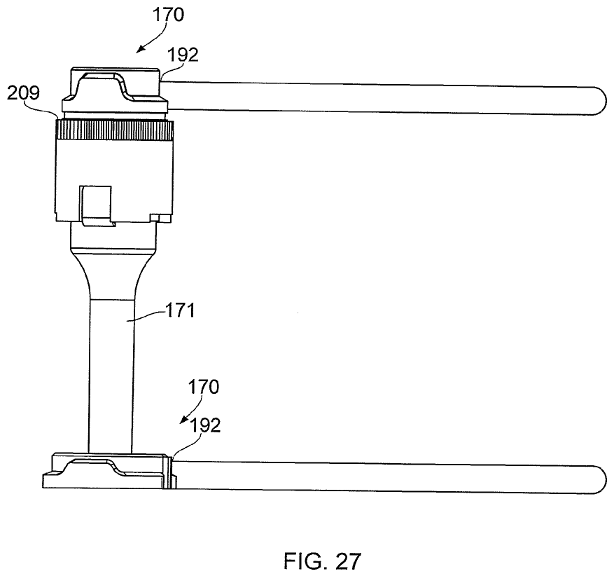

FIG. 27 is a side elevational view of two of the collection devices of FIG. 19 in use with a trocar.

Referring to the drawings and initially to FIGS. 1 to 5 thereof there is illustrated a collection device according to the invention indicated generally by the reference numeral 1 for minimising the dispersal of pathogens into the environment during insufflating of a cavity in a human or animal subject, in this case a human subject 3. The collection device 1 comprises a patch 5 for securing to the skin 6 of the subject 3 around but spaced apart from, an opening in the subject 3, for example, an incision 8 in an abdominal wall 9 of the subject 3, through which an access device, in this embodiment of the invention a trocar 10 extends into a cavity, in this case the peritoneal cavity 12 of the subject 3. The trocar 10 extends from a proximal end 14 to a distal end 15, and is of circular transverse cross-section having an instrument bore (not shown) extending therethrough of circular transverse cross-section, for accommodating an instrument therethrough to the peritoneal cavity 12 for carrying out an investigative or surgical procedure minimally invasively, for example, laparoscopically, while the peritoneal cavity is insufflated.

The patch 5 as will be described below is configured to sealably engage the abdominal wall 9 and the trocar 10, and to define with the trocar 10 and the adjacent portion of the skin 6 of the abdominal wall 9 an annular collection chamber 17 extending around the trocar 10 for collecting insufflating gas leaking from the peritoneal cavity 12 through the incision 8 between the abdominal wall 9 and the trocar 10. The patch 5, in this embodiment of the invention comprises an airtight flexible material impermeable to the insufflating gas, and in this case comprises a flexible plastics material. The patch 5 when viewed in plan is of circular shape, although, it will be readily apparent to those skilled in the art that the patch 5 may be of any suitable shape, for example, square shape, rectangular shape, triangular shape, hexagonal shape, or any other suitable shape. It will also be appreciated that the patch 5 may comprise any other suitable gas impermeable material, which is impermeable to the insufflating gas.

The patch 5 terminates in an outer peripheral edge 19, and an outer peripheral portion 20 extending around the patch 5 adjacent the outer peripheral edge 19 and is adapted for securing the patch to the skin 6 of the abdominal wall 9 of the subject 3 around the incision 8 but spaced apart radially outwardly therefrom. A securing means, in this embodiment of the invention a pressure sensitive adhesive 21 is coated onto an under-surface 22 of the outer peripheral portion 20 for releasably and sealably securing the patch 5 to the abdominal wall 9 around the outer peripheral edge 19 thereof.

An access opening 24 extends through the patch 5 spaced apart inwardly from the outer peripheral edge 19 for accommodating the trocar 10 therethrough. In this embodiment of the invention the access opening 24 is circular and is centrally located in the patch 5. The material of the patch 5 is such that an inner portion 23 thereof adjacent and extending around the access opening 24 is adapted to be sufficiently resilient to tightly and sealably engage the trocar 10 extending through the access opening 24, and to be sufficiently flexible to allow side-to-side tilting movement of the trocar 10.

An outlet port 25 extending through the patch 5, is adapted for coupling to a vacuum system 27 described in more detail below. The outlet port 25 communicates the collection chamber 17 with the vacuum system 27 for drawing insufflating gas from the collection chamber 17 leaking from the peritoneal cavity 12 through the incision 8 into the collection chamber 17.

A filter 29 for filtering out one or more of pathogens, bacteria, infections and viruses from the gases drawn through the outlet port 25 is coupled to the outlet port 25 between the outlet port 25 and the vacuum system 27. The filter 29 is adapted for filtering out particles of minimum size greater than 0.05 microns from the gases passing therethrough to the vacuum system 27, so that the gases from the vacuum system can be safely dispersed into the environment from the vacuum system 27 without pathogens, bacteria, infections and viruses.

A support element, in this embodiment of the invention a support framework 30 is provided for supporting the patch 5 to form the collection chamber 17 and to prevent the patch 5 collapsing when a vacuum is applied to the outlet port 25. In this embodiment of the invention the support framework 30 comprises a lower framework 31 and an upper framework 32 spaced apart above the lower framework 31. Both the lower and upper frameworks 31 and 32 comprise circular ring frames 33 and 34, respectively, of a suitable medical grade stainless steel wire material. The diameter of the lower ring frame 33 is greater than the diameter of the upper ring frame 34. A plurality of circumferentially spaced apart struts 35 extend radially and downwardly from the upper ring frame 34 to the lower frame 33 for supporting the upper ring frame 34 above the lower frame 33. The struts are of a suitable medical grade stainless steel wire material similar to that of the lower and upper ring frames 33 and 34. The diameter of the lower ring frame 33 is less than the inner diameter of the outer peripheral portion 20 of the patch 5 so that the outer peripheral portion 20 of the patch 5 extends radially outwardly beyond the lower ring frame 33. The diameter of the upper ring frame member 34 is sufficiently greater than the diameter of the access opening 24 extending through the patch 5 in order that the inner portion of the patch 5 extending between the access opening 24 and the upper ring frame 34 is free to tightly and sealably engage the trocar 10 extending therethrough. The inner portion 23 of the patch 5 is sufficiently flexible to permit the side-to-side tilting movement of the trocar 10. The patch 5 may be secured to the support framework 30, or may be provided to merely drape over the support framework 30.

In this embodiment of the invention the access opening 24 and the lower and upper ring frames 33 and 34 are concentric with the outer peripheral edge 19 of the patch 5.

Referring now to FIG. 5 the vacuum system 27 is illustrated in block representation and comprises a vacuum pump 37 connected to an outlet port 38 from the filter 29 by a vacuum line 39, and is configured to draw a vacuum on the outlet port 38 of the filter 29 and in turn to draw a vacuum on the outlet port 25 from the patch 5. Filtered gases drawn from the outlet port 38 of the filter 29 by the vacuum pump 37 are discharged safely into the environment from an exhaust port 40 of the vacuum pump 37. The filter 29 may be connected directly into the outlet port 25 from the patch or may be connected to the outlet port 25 through a conduit 41 as illustrated in FIG. 5. The vacuum pump 37 is adapted to generate a vacuum of pressure in the range of 150 mmHg to 300 mmHg below atmospheric pressure for applying to the outlet port 25 of the collection device 1 through the filter 29.

In use, if the patch 5 is not secured to the support framework 30, the support framework 30 is located on the abdominal wall 9 of the subject 3 substantially concentrically with the incision 8 formed in the abdominal wall 9. The trocar 10 is then urged through the access opening 24 extending through the patch 5, and in turn is passed through the support framework 30 and through the incision 8 formed in the abdominal wall 9, and in turn is urged into the peritoneal cavity 12. The patch 5 is then manoeuvred on the trocar 10, so that the patch 5 drapes over and is supported on the support framework 30. The outer peripheral portion 20 of the patch 5 is then secured to the skin 6 of the abdominal wall 9 of the subject by the pressure sensitive adhesive 21 in order to form the collection chamber 17 between the patch 5, the abdominal wall 9 and the trocar 10. The outlet port 25 from the patch 5 is then connected to the vacuum system 27 through the filter 29.

If on the other hand, the patch 5 is secured to the support framework 30, the trocar 10 is initially entered through the access opening 24 in the patch 5. The trocar 10 is then urged into the incision 8 in the abdominal wall 9, and as the trocar 10 is being urged through the incision 8, the patch 5 with the support framework 30 attached thereto is positioned on the abdominal wall 9 substantially concentrically with the incision 8, and the outer peripheral portion 20 of the patch 5 is then secured to the skin 6 of the abdominal wall 9 by the pressure adhesive 21.

When insufflating of the peritoneal cavity 12 of the subject 3 commences, the vacuum from the vacuum system 27 is applied to the outlet port 25 from the patch 5 through the filter 29. Insufflating and other gases leaking through the incision 8 in the abdominal wall 9 between the abdominal wall 9 and the trocar 10 into the collection chamber 17 are drawn from the collection chamber 17 through the outlet port 25 thereof, and in turn through the filter 29 for filtering thereof. Pathogens and other particulate matter including bacteria, infections and viruses are collected in the filter 29 for subsequent removal and safe disposal. The clean filtered gases are then dispersed through the exhaust port 40 of the vacuum pump 37 safely into the environment.

It will of course be appreciated that instead of connecting the collection device 1 through the filter 29 to the vacuum system 27, the collection device 1 may be connected to a vacuum system provided in, for example, an operating theatre, or in other locations where the procedure is being carried out on a subject. In which case the collection device would be connected to the vacuum system of the operating theatre through the filter 29, or if the vacuum system of the operating theatre included a filtration system the collection device 1 may be connected directly into the vacuum system of the operating theatre.

It is also envisaged that in some embodiments of the invention a portion of the patch, for example, an intermediate portion thereof between the outer peripheral portion 20 and the inner portion 23 and extending around the inner portion 23 thereof may be provided in the form of a self-supporting material, for example, a semi-rigid or a rigid material, which would be shaped to form the collection chamber 17 with the trocar 10 and the skin 6 of the subject. In which case, once the patch or the intermediate portion thereof would be self-supporting, and would remain self-supporting under the vacuum applied through the outlet port 25, the support framework may be omitted.

It will of course be appreciated that any other suitable support framework besides that described may be used.

Referring now to FIGS. 6 to 11 there is illustrated an access device also according to the invention for accommodating an instrument through an opening in a human or animal subject, and for minimising the dispersal of pathogens into the environment during insufflating of a vessel, lumen or cavity in the digestive tract, or accessible through the digestive tract of a subject. The access device in this embodiment of the invention comprises a mouth entry device, which is according to the invention, and is indicated generally by the reference numeral 45 for accommodating an instrument, for example, an endoscope (not shown) orally into, for example, the digestive tract of the subject. The mouth entry device 45 typically is provided for accommodating an endoscope or other instrument orally into a vessel, lumen or cavity in the digestive tract or accessed through the digestive tract in which an investigative or surgical procedure is being carried out minimally invasively, for example, laparoscopically, and in which the vessel, lumen or cavity, typically is being insufflated.

The mouth entry device 45 comprises a tubular member 47 of oval type transverse cross-section extending between a proximal end 48 and a distal end 49 and defining an inner surface 50 and an outer surface 51. The inner surface 50 defines an instrument bore 53 of the trocar 10 extending through the tubular member 47 from the proximal end 48 to the distal end 49 for accommodating the instrument (not shown) through the mouth entry device 45. The instrument bore 53 is of transverse cross-sectional shape substantially similar to that of the outer transverse cross-section of the tubular member 47, but of reduced dimensions. The tubular member 47 defines the inner surface 50 and a part of the outer surface 51 of the mouth entry device 45.

A proximal flange 55 extends radially outwardly and circumferentially around the tubular member 47 adjacent the proximal end 48, and a distal flange 56 extends radially outwardly and circumferentially around the tubular member 47 adjacent the distal end 49 thereof. A pair of side flanges 58 extend radially outwardly from the proximal flange 55 on respective opposite sides thereof. The proximal flange 55 defines a first surface 59 and a second surface 60. The second surface 60 merges into the part of the outer surface 51 formed by the tubular member 47 through a radiused surface 61 extending around the mouth entry device 45, and defines with the part of the outer surface 51 of the tubular member 47 and the radiused surface 61, the outer surface 51 of the mouth entry device 45. When the mouth entry device 45 is inserted into the mouth of a subject, the proximal flange 55 remains outside the mouth of the subject, with the second surface 60 abutting the face of the subject around the mouth thereof. The distal flange 56 is located in the mouth of the subject behind the upper and lower teeth of the subject, so that the subject can bite on the outer surface 51 of the tubular member 47 with his or her teeth. This aspect of a mouth entry device will be well known to those skilled in the art.

A collection chamber, in this embodiment of the invention an annular collection chamber 63 is located in the mouth entry device 45 adjacent the proximal flange 55. The collection chamber 63 is located between the inner surface 50 and the outer surface 51 of the mouth entry device 45 adjacent the radiused surface 61, and extends circumferentially completely around the instrument bore 53 and is spaced apart therefrom within the tubular member 47.

An outlet bore 64 extends radially outwardly from the collection chamber 63 through the proximal flange 55 and one of the side flanges 58 and terminates in an outlet port 65 extending from the corresponding side flange 58. The outlet port 65 is adapted for coupling to a vacuum system 66 described below for applying a vacuum to the collection chamber 63 for evacuating gases from the collection chamber 63.

A plurality of first inlet ports 67, which are spaced apart around the collection chamber 63, extend radially outwardly from the collection chamber 63 and communicate with the collection chamber 63. The first inlet ports 67 terminate in the outer surface 51, namely, the radiused portion 61 thereof. The first inlet ports 67 are substantially equi-spaced apart circumferentially around the outer surface 51, and are adapted to draw gases including gases being exhaled by the subject and insufflating gases leaking through the mouth of the subject into the collection chamber 63 in response to a vacuum being applied to the outlet port 65.

A plurality of second inlet ports 69 spaced apart along the collection chamber 63 extend radially inwardly from the collection chamber 63 and communicate with the collection chamber 63. The second inlet ports 69 terminate in the instrument bore 53 in the inner surface 50 of the tubular member 47. The second inlet ports 69 are substantially equi-spaced apart circumferentially around the inner surface 50, and are adapted to draw gases passing through the instrument bore 53 being exhaled by the subject and insufflating gases leaking through the instrument bore 53 into the collection chamber 63 in response to a vacuum being applied to the outlet port 65.

Turning now to the vacuum system 66, and referring in particular to FIG. 11, the vacuum system 66 is substantially similar to the vacuum system 27 of the collection device 1 described with reference to FIGS. 1 to 5, and similar components are identified by the same reference numerals. In this embodiment of the invention a filter 70 which is similar to the filter 29 of the collection device 1 is coupled to the outlet port 65 for filtering out one or more of pathogens, bacteria, infections and viruses from the gasses drawn through the outlet port 65. The filter 70 is coupled to the outlet port 65 either directly or through the conduit 41. A vacuum is applied to the filter 70 through the vacuum line 39 by the vacuum pump 37 for drawing the gases from the collection chamber 63 through the outlet port 65 and in turn through the filter 70. The clean filtered gases are dispersed safely into the environment through the exhaust port 40 of the vacuum pump 37. The vacuum drawn on the first and second inlet ports 67 and 69 is a relatively light vacuum in order to avoid any danger of interfering with or impeding breathing by the subject. In this embodiment of the invention the vacuum pump 37 is adapted to generate a vacuum of pressure in the range of 150 mmHg to 300 mmHg which is applied through the filter 70 to the outlet port 65 from the collection chamber 63

In use, with the mouth entry device 45 inserted in the mouth of the subject with the proximal flange 55 abutting the face of the subject around the mouth of the subject and with the teeth of the subject biting on the outer surface 51 of the tubular member 47, an endoscope or other instrument or instruments, including an insufflating tube is passed through the instrument bore 53 of the mount entry device 45. The vacuum pump 37 is activated to apply a vacuum to the collection chamber 63 through the filter 70 and the outlet port 65 for in turn drawing a vacuum on the first and second inlet ports 67 and 69, for in turn drawing gases including insufflating gas and gases being exhaled orally by the subject into the collection chamber 63. The gases are then drawn from the collection chamber 63 through the outlet port 65 and in turn are filtered through the filter 70 and clean filtered gases are dispersed safely into the environment through the exhaust port 40 of the vacuum system 66.

It will of course be appreciated that in the event of a vacuum system being available in an operating theatre or other area where a procedure using the mouth entry device 45 is being carried out, the filter 70 may be coupled to such a vacuum system. Additionally, where such a vacuum system exists with a filtration system, the filter 70 may be omitted.

Accordingly, the mouth entry device 45 according to the invention at least minimises and in general prevents the dispersal of pathogens into the environment during insufflating of a cavity in a subject.

Referring now to FIGS. 12 and 13 there is illustrated an access device according to another embodiment of the invention for accommodating an instrument into a cavity in a human or animal subject through an opening in the subject and for minimising the dispersal of pathogens into the environment during insufflating of the cavity. In this embodiment of the invention the access device comprises a trocar according to the invention indicated generally by the reference numeral 75. The trocar 75 is illustrated in FIG. 12 partly in cross-section extending through an opening, namely, an incision 77 in the abdominal wall 78 into the peritoneal cavity 79. The trocar 75 comprises a tubular member 80 of circular transverse cross-section extending between a proximal end 82 and a distal end 83 which extends into the peritoneal cavity 79. The proximal end 82 terminates in a proximal housing 85, within which a valve (not shown), for example, a duckbill valve, a multi-leaf valve, a cross-slit valve, or other suitable valving arrangement is located for sealably and slideably engaging an instrument, for example, a laparoscope extending through the trocar 75. Such valves for sealably and slideably engaging a laparoscope or other such instrument being entered into a subject through a trocar will be well known to those skilled in the art, and further description should not be required.

The tubular member 80 defines an inner surface 86 and an outer surface 87. The inner surface 86 defines an instrument bore 89 for accommodating an instrument therethrough.

A collection chamber, in this embodiment of the invention an annular collection chamber 90 extends completely around and within the tubular member 80 adjacent the proximal end 82 thereof between and spaced apart from the inner and outer surfaces 86 and 87 and around and spaced apart from the instrument bore 89. The collection chamber 90 is substantially similar to the collection chamber 63 formed in the mouth entry device 45 described with reference to FIGS. 6 to 11, and similar components are identified by the same reference numerals.

An outlet bore 64 extends from the collection chamber 90 through a base wall 91 of the proximal housing 85 and terminates in an outlet port 65 adapted for coupling to a vacuum system 92 for applying a vacuum to the collection chamber 90. The vacuum system 92 is similar to the vacuum system 66 of the mouth entry device 45, and similar components are identified by the same reference numerals.

A plurality of first inlet ports 67 communicate with and extend radially outwardly from the collection chamber 90 and terminate in the outer surface 87 of the tubular member 80 adjacent the proximal end 82 thereof, for drawing gases escaping through the incision 77 in the abdominal wall 78 between the trocar 10 and the abdominal wall 78 into the collection chamber 90 in response to a vacuum being applied to the outlet port 65. The first inlet ports 67 terminate in the outer surface 87 of the tubular member 80 adjacent but spaced apart distally from the proximal housing 85 in a short portion 94 of the trocar 75, which in use is located between the base wall 91 of the proximal housing 85 and the abdominal wall or other wall of a subject through which the trocar 75 extends, in order to maximise the collection of gases escaping through the incision 77. However, it is envisaged that the first inlet ports 67 may terminate in the base wall 91 of the proximal housing 85 adjacent the tubular member 80 or slightly spaced apart therefrom. In this embodiment of the invention the first inlet ports 67 are substantially equi-spaced apart around the tubular member 80.

A plurality of second inlet ports 69 communicate with and extend radially inwardly from the collection chamber 90, and terminate in the inner surface 86 defining the instrument bore 89 for drawing insufflating gases and other gases passing through the instrument bore 89 from the peritoneal cavity 79 into the collection chamber 90 in response to a vacuum being applied to the outlet port 65. The second inlet ports 69 are substantially equi-spaced apart circumferentially around the instrument bore 89.

A filter 93 is coupled to the outlet port 64 for filtering gases drawn from the collection chamber 90 through the outlet port 65 by the vacuum system 92. In this embodiment of the invention the filter 93 is similar to the filter 70 and is adapted for filtering out one or more of pathogens, bacteria, infections and viruses. The filter 93 may be coupled directly to the outlet port 65 from the collection chamber 90 or may be coupled to the outlet port 65 through the conduit 41. A vacuum is applied by the vacuum pump 37 to the collection chamber 90 through the vacuum line 39, the filter 70 and the outlet port 65. Accordingly, when a vacuum is applied to the collection chamber 90, gases escaping from the peritoneal cavity 79 through the incision 77 are drawn through the first inlet ports 67 into the collection chamber 90, and gases escaping from the peritoneal cavity 79 through the instrument bore 89 of the trocar 75 are drawn through the second inlet ports 69 into the collection chamber 90. The gases in the collection chamber 90 are then drawn through the outlet port 65 and in turn the filter 93, where pathogens, bacteria, infections and viruses are filtered from the gases by the filter 93, and clean filtered gases are then safely dispersed into the environment through the exhaust port 40 of the vacuum pump 37. The vacuum pump 37 is adapted to generate a vacuum of pressure in the range of 150 mmHg to 300 mmHg below atmospheric pressure for applying through the filter 93 to the outlet port 65 from the collection chamber 90.

In use, with the trocar 75 entered into the peritoneal cavity 79 through the incision 77 in the abdominal wall 78 of the subject, and with the proximal housing 85 located adjacent but spaced apart from the abdominal wall 78 with the first inlet ports 67 close to but spaced apart from the abdominal wall 78 on the external side thereof, and with the trocar 75 coupled to the vacuum pump 37 through the filter 93, the trocar 75 is ready for use. Any gases passing through the instrument bore 89 from the peritoneal cavity 79 are drawn by the vacuum through the second inlet ports 69 into the collection chamber 90, and any gases escaping through the incision 77 in the abdominal wall 78 are drawn by the vacuum through the first inlet ports 67 into the collection chamber 90. The gases in the collection chamber 90 are drawn through the outlet port 65 and in turn through the filter 93 where pathogens, bacteria, infections and/or viruses are filtered from the gases and clean filtered gases are safely dispersed into the environment through the exhaust port 40 of the vacuum pump 37.

Accordingly, the trocar 75 according to the invention at least minimises and in general prevents the dispersal of pathogens into the environment during insufflating of a cavity in a subject.

It is envisaged that while the collection chamber 90 of the trocar 75 has been described as being formed in the tubular member 80 adjacent the proximal end thereof, in some embodiments of the invention where it is expected that the proximal end of the trocar will be spaced apart some distance from the abdominal wall, or other wall of the subject through which the trocar is being entered into the appropriate cavity of the subject, in such cases, the collection chamber may be located spaced apart from the proximal end of the trocar at a distance, so that the collection chamber and the first inlet ports would terminate in the outer surface 87 of the tubular member 80 at a position such that when the trocar is entered into the appropriate cavity through the incision or other opening formed in the wall of the subject, the first inlet ports would be located externally of the wall of the subject, but sufficiently close to the wall of the subject to draw in the gases leaking through the incision or other opening between the trocar and the wall of the subject.

Referring now to FIGS. 14 and 15 there is illustrated apparatus according to the invention indicated generally by the reference numeral 95 for minimising dispersal of gases escaping from one or more openings in a subject 96 through which respective access devices extend into a cavity of the subject 96 being insufflated, and in which an investigative or surgical procedure is being carried out minimally invasively. The apparatus 95 comprises a plurality of collection elements 97 which are configured in an endless daisy-chain formation and are connected by suitable connecting elements 98, for example, tethers or chains. In this embodiment of the invention the collection elements 97 are illustrated placed on the skin 100 of the abdominal wall 101 of a subject around a site in which three openings, in this case incisions 103 are formed through the abdominal wall 101 into the peritoneal cavity (not shown) of the subject 96 for accommodating three trocars 106 therethrough into the peritoneal cavity. The trocars 106 are provided for accommodating instruments therethrough into the peritoneal cavity of the subject, such as a laparoscope for carrying out a procedure laparoscopically and other instruments, as well as insufflating gas for insufflating the peritoneal cavity, as will be well known to those skilled in the art. The collection elements as will be described below are adapted to draw insufflating and other gases leaking through the incisions 103 between the respective trocars 106 and the abdominal wall 101.

Each collection element 97 comprises a base wall 108 and a top wall 109 spaced apart above the base wall 108. A first wall, namely, a front wall 110 extends upwardly from the base wall 108 and joins the base wall 108 with the top wall 109. A second wall, in this case comprising a rear wall 112 and a pair of spaced apart side walls 114 extend upwardly from the base wall 108 to the top wall 109 joining the top wall 109 to the base wall 108. The rear wall 112 is spaced apart from the front wall 110 and the side walls 114 are spaced apart from each other and join the front end wall 110 adjacent its opposite ends 115 to the rear wall 112. The base wall 108, the top wall 109, the front wall 110, the rear wall 112 and the side walls 114 together define a collection chamber 117.

An outlet port 118 extending from the rear wall 112 of each collection element 97 communicates with the collection chamber 117 thereof and is adapted for connecting the collection chamber 117 of the corresponding collection element 97 to a vacuum system 119, for applying a vacuum to the collection chamber 117, as will be described in detail below.

A plurality of spaced apart inlet ports, in this case three inlet ports 120 extend through the front wall 110 of each collection element 97, and communicate with the collection chamber 117 thereof, for drawing gases escaping through the incisions 103 in the abdominal wall 101 into the collection chamber 117 of the corresponding collection element 97 when a vacuum is applied to the collection chamber 117.

A manifold, in this embodiment of the invention an endless conduit 122 comprises a plurality of inlet ports 123 spaced apart longitudinally along the conduit 122. The inlet ports 123 communicate with an endless bore (not shown) extending through the endless conduit 122. The inlet ports 123 of the endless conduit 122 are connected by connecting conduits 125 to the outlet ports 118 of the respective collection elements 97. A main outlet port 127 extending from the endless conduit 122 and communicating with the endless bore (not shown) of the endless conduit 122 connects the endless conduit 122 with the vacuum system 119 through a filter 116 for applying a vacuum to the collection chambers 117 in the collection elements 97 through the connecting conduits 125. The filter 116 is similar to the filter 29 of the collection device 1 described with reference to FIGS. 1 to 6. The vacuum system 119 is also similar to the vacuum system 27 of the collection device 1, and similar components are identified by the same reference numerals. The vacuum pump 37 is adapted to generate a vacuum of pressure in the range of 150 mmHg to 300 mmHg for applying through the filter 116 to the main outlet port 127 from the endless conduit 122.

A securing means in this embodiment of the invention a releasable pressure sensitive adhesive 129 is coated onto the base wall 108 of each collection element 97 for releasably securing the collection elements 97 onto the skin 100 of the subject.

In use, when the apparatus 95 is to be used in conjunction with a procedure to be carried out laparoscopically in the peritoneal cavity, and the peritoneal cavity is being insufflated, the collection elements 97 in the endless daisy-chain formation are placed on the skin 100 of the abdominal wall 101 of the subject 96 around the site in which one or more trocars 106 are being entered into the peritoneal cavity through the corresponding incisions 103. The collection elements 97, when being placed on the skin 100 of the subject 96 are releasably bonded to the skin 100 of the subject by the pressure sensitive adhesive 129 with the inlet ports 120 facing inwardly towards the site and the trocars 106. The endless conduit 122 connected to the collection elements 97 by the connecting conduits 125 is then placed on the abdominal wall 101 of the subject with the endless conduit 122 extending around the collection elements 97. The outlet port 127 of the endless conduit 125 is then connected through the filter 116 to the vacuum system 119. The filter 116 may either be connected directly into the outlet port 127 of the endless conduit 122, or may be connected through the conduit 41 to the outlet port 127.

The vacuum pump 37 is activated, and a vacuum is applied through the filter 116, the endless conduit 122 and the connecting conduits 125 to the collection chambers 117 of the collection elements 97. Insufflating and other gases escaping through the incisions 103 is then drawn through the inlet ports 120 of the collection elements 97 into the collection chambers 117 thereof, and in turn through the endless conduit 122 through the filter 116 where any pathogens, including bacteria, infections and/or viruses and other particulate matter are filtered out of the gases, which are then safely discharged through the exhaust port 40 of the vacuum pump 37.

On completion of the procedure the collection elements 97 are removed along with the endless connecting conduit 125.

While the collection elements 97 have been described as being connected in an endless daisy-chain formation, the collection elements 97 may be connected in a non-endless daisy-chain formation. Similarly, while the endless conduit 122 has been described as being provided as an endless conduit, the conduit may be provided as a non-endless conduit.

Referring now to FIGS. 16 to 18, there is illustrated an access device according to another embodiment of the invention for accommodating an instrument through an opening in a human or animal subject into a cavity of the subject during a minimal invasive procedure, and for minimising dispersion of Pathogens into the environment during insufflating of the cavity of the subject. In this embodiment of the invention the access device comprises a transanal entry device, which is according to the invention, and is indicated generally by the reference numeral 140 for accommodating an instrument, for example, an colonoscope (not shown) anally into the rectum, the colon or the lower digestive tract of the subject.

The transanal entry device 140 is substantially similar to the mouth entry device 65, with the exception that the transanal entry device 140 is provided without a distal flange, and without the two side flanges extending from the proximal flange of the mouth entry device. Accordingly, the transanal entry device 140 comprises a tubular member 142 of circular transverse cross-section extending between a proximal end 143 and a distal end 144, and defines an inner surface 145 and an outer surface 146. The inner surface 145 defined by the tubular member 142 defines an instrument bore 148 of circular transverse cross-section extending through the tubular member 142 from the proximal end 143 to the distal end 144. The tubular member 142 is adapted for extending through the anus into the rectum for accommodating instruments through the instrument bore 148 thereof, to the distal end 144 for example, a colonoscope, an endoscope and other surgical instruments as would be required for carrying out a procedure in the rectum, the colon or the lower end of the digestive tract of a subject, or in a lumen, vessel or cavity accessed through the rectum, the colon or lower digestive tract.

A circular proximal flange 150 extends circumferentially around and radially outwardly from the tubular member 142 adjacent the proximal end 143 thereof for engaging the subject externally of but adjacent the anus. The proximal flange 150 defines a first surface 152 and a second surface 154. The second surface 154 of the proximal flange 150 merges with the outer surface 146 of the tubular member 142 through a radiused surface 156 extending around the tubular member 142. The second surface 154 and the radiused surface 156 defines with the outer surface 146 an outer surface 157 of the transanal entry device 140.

An annular collection chamber 158 extends completely circumferentially around and within the tubular member 142 and circumferentially around the instrument bore 148, and is located between the inner surface 145 and the outer surface 157 of the transanal entry device 140 adjacent the radiused surface 156 and adjacent the proximal flange 150. An outlet bore 160 communicating with and extending from the collection chamber 158 through the proximal flange 150 terminates in an outlet port 161 extending outwardly of the proximal flange 150. The outlet port 161 is adapted for connecting to a vacuum system (not shown) through a filter (also not shown) for drawing a vacuum in the collection chamber 158. The vacuum system to which the outlet port 161 of the transanal entry device 140 is connected is similar to the vacuum system 66 to which the outlet port 65 of the mouth entry device 45 is connected. Additionally, the filter (not shown) through which the outlet port 161 is connected to the vacuum system (not shown) is similar to the filter 70 through which the outlet port 65 of the mouth entry device 45 is connected to the vacuum system 66.

A plurality of first inlet ports 163 communicating with the collection chamber 158 and extending radially outwardly therefrom terminate in the radiused surface 156 of the transanal entry device 140 for drawing gases, including insufflating gases escaping through the anus of a subject between the anus of a subject and the tubular member 142 into the collection chamber 158 in response to a vacuum being applied to the outlet port 161. The first inlet ports 163 are substantially equi-spaced apart circumferentially around the radiused surface 156 of the transanal entry device 140.

A plurality of second inlet ports 165 communicating with the collection chamber 158 extend radially inwardly from the collection chamber 158 and terminate in the inner surface 145 of the transanal entry device 140 for drawing gases including insufflating gases escaping from the rectum through the instrument bore 148 in response to a vacuum being applied to the outlet port 161. The second inlet ports 165 are substantially equi-spaced apart around the instrument bore 148.

In use, the transanal entry device 140 is inserted, distal end 144 first, into the anus of a subject until the proximal flange 150 is located close to but spaced apart from the anus of the subject with the first inlet ports 163 located adjacent but spaced apart externally from the anus, so that when a vacuum is applied to the outlet port 161 gases including insufflating gases, which escape from the anus between the tubular member 142 and the anus, are drawn through the first inlet ports 163 into the collection chamber 158, and thereafter through the outlet port 161. The outlet port 161 is then connected to the vacuum system (not shown) through the filter (not shown). The filter (not shown) may be connected directly to the outlet port 161 or may be connected to the outlet port 161 through the conduit 41. In this embodiment of the invention the vacuum pump of the vacuum system is adapted to generate a vacuum in the range of 150 mmHg to 300 mmHg below atmospheric pressure for applying through the filter (not shown) to the outlet port 161.

On activation of the vacuum system, gases, including insufflating gases escaping from the rectum through the anus and through the instrument bore 148 are drawn through the first and second inlet ports 163 and 165, respectively, into the collection chamber 158. The gases drawn into the collection chamber 158 are drawn from the collection chamber 158 through the outlet port 161 and in turn through the filter. The filter (not shown) removes pathogens, including bacteria, infections and viruses from the gases, and the filtered gases are then safely dispersed into the environment through the exhaust port 40 of the vacuum pump 37 of the vacuum system.

Referring now to FIGS. 19 to 28 there is illustrated a collection device according to the invention and indicated generally by the reference numeral 170 for minimising the dispersal of pathogens into the environment during insufflation of a lumen, vessel or cavity in a human or animal subject. The collection device 170 is particularly suitable for locating around a trocar or other access device adjacent an incision or opening in the body of a human or animal subject through which the trocar or access device extends into a cavity being insufflated in the human or animal body. The collection device 170 is provided to extend around the access device, in this case a trocar 171 adjacent the incision (not shown) to collect any gases including insufflating gases escaping through the incision around the trocar 171 in order to prevent or at least minimise the escape of such gases including insufflating gases dispersing into the environment.

The collection device 170 comprises a pair of arcuate members 172, which in this case are of semi-circular shape and extend from a first end 174 to a second end 175. The arcuate members 172 are hingedly coupled by a hinge 177 adjacent the first ends 174 thereof. The arcuate members 172 are hingeable about the hinge 177 from a closed state illustrated in FIG. 19 with the second ends 175 abutting each other and the arcuate members 172 defining an access bore 178 for accommodating a trocar 171 therethrough, to an open state (not shown) with the second ends 175 of the arcuate members 172 spaced apart from each other sufficiently in order to accommodate the trocar 171 radially between the second ends 175 into the access bore 178.

Each arcuate member 172 comprises a lower arcuate member 180 of U-shape transverse cross-section defining an arcuate channel 181 extending through the lower arcuate member 180 from the first end 174 to the second end 175. An upper arcuate member 182 closes the channel 181 to form a collection chamber 184 in the corresponding arcuate member 172. The collection chambers 184 in the respective arcuate members 172 communicate with each other through the first and second ends 174 and 175 of the arcuate members 172 when the arcuate members are in the closed state. A pair of spaced apart legs, namely, an inner leg 186 and an outer leg 187 extend downwardly from the upper arcuate member 182 into the channel 181 for securing the upper arcuate member 182 to the lower arcuate member 180. An inwardly extending engagement ring 189 extends inwardly from the inner leg 186 of the upper arcuate member 182 for engaging a retaining ring 190 extending from an inner leg 191 of the lower arcuate member 180 into the channel 181 for securing the upper arcuate member 182 to the lower arcuate member 180.

An outlet port 192 extends outwardly from an outer leg 193 of the lower arcuate member 180 of one of the arcuate members 172, and communicates with the collection chamber 184 in that arcuate member 172. The outlet port 192 is adapted for coupling to a vacuum system through a filter, for example, for coupling to the vacuum system 27 of the collection device 1 described with reference to FIGS. 1 to 5, whereby the outlet port 192 would be connected to the vacuum system 27 through a filter similar to the filter 29 of the collection device 1.

A plurality of inlet ports 195 are located in the inner leg 191 of the lower arcuate member at spaced apart intervals around each arcuate member 172. The inlet ports 195 communicate with the collection chamber 184 and with the access bore 178, so that when the arcuate members 172 are in the closed state defining the access bore 178, any gases including insufflating gases in or passing through the access bore 178 are drawn from the access bore 178 through the inlet ports 195 into the respective collection chambers 184 under vacuum when a vacuum is applied to the outlet port 192. The gases are drawn from the collection chambers 184 of the respective arcuate members 172 through the outlet port 192 and the filter, for example, the filter 29 where any pathogens, including bacteria, infections, viruses and other particulate matter are filtered from the gases, and the filtered gases are then safely dispersed into the environment from the vacuum system 27.

A releasable fastening means, in this embodiment of the invention a releasable clip 198 comprising a pair of interengageable complementary formations, namely, a clasp 199 which is located on the second end 175 of one of the arcuate members 172, and a receiving recess 200 which is located on the second end 175 of the other one of the arcuate members 172 whereby the clasp 199 is adapted to engage the receiving recess 200 with a releasable snap-fit action.

In this embodiment of the invention a closure film 202 is secured between the upper arcuate members 182 and the lower arcuate members 180 of the respective arcuate members 172, for closing the access bore 178 adjacent the top thereof. A central opening 203 is formed in the closure film 202 for accommodating the trocar 171 therethrough into the access bore 178. A slit 204 extends radially through the closure film 202 from the central opening 203 to the second ends 175 of the arcuate members 172. The closure film 202 comprises an elastic material to permit hinging of the arcuate members 172 from the closed state to the open state for accommodating the trocar radially into the access bore 178 between the second ends 175 of the respective arcuate members 172. The closure film 202 forms a seal between the closure device 170 and the trocar 171 extending through the access bore 178 in order to minimise any gases leaking from the bore 178, and acts to urge the arcuate members 172 into the closed state.

A securing means, in this embodiment of the invention a pressure sensitive adhesive coating 206 is coated onto a base plate 205 of the lower arcuate member 180 of each arcuate member 172 for releasably securing the collection device 170 to either the skin of a subject around the incision or opening through which the trocar 171 or other access device is extending, or alternatively, for releasably securing the collection device 170 around an instrument port of a trocar or other access device for collecting gases escaping through the instrument port of the trocar or other access device.

Referring now to FIG. 27 two of the collection devices 170 are illustrated in use. The first one of the collection devices 170a is located secured to the skin of the subject around an incision (not shown) through which the trocar 171 extends into a cavity, for example, the peritoneal cavity, whereby the incision is formed in the abdominal wall of the subject. When a vacuum is applied to the outlet port 192 of the collection device 170a any gases escaping from the incision around the trocar 171 are drawn from the access bore 178 through the inlet ports 195 into the collection chamber 184, and in turn through the outlet port 192 and the filter where the gases are filtered for removing pathogens, including bacteria, infections, viruses and other particulate matter prior to being dispersed safely into the environment.

The collection device 170b is illustrated located adjacent an instrument port 209 of the trocar 171 for collecting any gases which may escape through the instrument bore (not shown) of the trocar 171.

While a specific fastening means for securing the second ends of the arcuate members together has been described, any other suitable fastening means may be provided.

While the collection device has been described as comprising a securing means for securing the device to, for example, the skin of a subject, in some embodiments of the invention it is envisaged that the securing means may be omitted. It is also envisaged that where a securing means is provided for securing the collection device to the skin of a subject or to an instrument port of an access device, any other securing means may be provided, for example, clips or the like.

While the collection device has been described a comprising a closure film, in some embodiments of the invention it is envisaged that the collection device may be provided without such a closure film.

While the apparatus 95 described with reference to FIGS. 14 and 15 has been illustrated as comprising eleven collection elements, the apparatus 95 may be provided with any suitable number of collection elements 97, greater or less than eleven, and the number of collection elements in the daisy-chain formation will be dependent on the size of the site through which one or more trocars are entered into the cavity being insufflated through the wall of the subject. It will also be appreciated that while the collection elements have been described as being of a specific shape and construction, the collection elements may be provided in any other shape and construction. Indeed, it is envisaged in some embodiments of the invention that instead of providing each collection element with a plurality of inlet ports, a single elongated inlet slot could be formed in the front wall of each collection element.

It is also envisaged that in the collection device described with reference to FIGS. 1 to 5, the patch may be permanently secured to the trocar, and would extend around the trocar, in the form of a skirt, the outer periphery of which would be configured to be secured to the subject around but spaced apart from the incision through which the trocar extends into the subject. In cases where the skirt formed by the patch of the device of FIGS. 1 to 5 is provided with a support framework, the support framework may or may not be secured to the trocar. It is also envisaged that the patch and the support framework may be permanently secured to the trocar, or may be integrally formed with the trocar. It is also envisaged that in cases where the patch is permanently secured to the trocar extending around the trocar, the patch may be of a flexible material as described with reference to FIGS. 1 to 5, or may be of a self-supporting material such as a semi-rigid material with an outer peripheral portion extending around the outer peripheral edge thereof being flexible for facilitating securing of the patch to the subject.

While the collection device described with reference to FIGS. 1 to 5 has been described in conjunction with a trocar, where the procedure is being carried out as a laparoscopic procedure, it is envisaged that the device of FIGS. 1 to 5 may be used in conjunction with an endoscope in a procedure being carried out endoscopically.

It will be appreciated that while the respective collection chambers of the trocar, the mouth entry device and the transanal entry device have been described as being annular collection chambers extending completely around the instrument bore of the trocar and the mouth and transanal entry devices, in some embodiments of the invention it is envisaged that the collection chambers may not extend completely around the trocar, the mouth or transanal entry devices, and it is also envisaged that in some embodiments of the invention a number of circumferentially spaced apart collection chambers may be provided.

While the collection device, access devices, mouth and transanal entry devices, trocar and apparatus have been described as being adapted for coupling to a specific type of vacuum system, it is envisaged that the collection device, access devices, mouth and transanal entry devices, trocar and apparatus may be configured for coupling to any suitable vacuum system. It is also envisaged that the collection device, access devices, mouth and transanal entry devices, trocar and apparatus may be configured for coupling to a vacuum system of a building, for example, an operating theatre. Where such an operating theatre vacuum system is provided with a filter, the filters, through which of the collection device, access devices, mouth and transanal entry devices, trocar and apparatus are connected to the vacuum systems, may be omitted. While the vacuum systems have been described as producing vacuums of a specific range, it will be readily apparent to those skilled in the art that the vacuum pumps of the vacuum systems may be adapted to produce vacuum at other ranges of values. Furthermore, it will be appreciated that while a specific range of values of vacuum have been described for applying to the collection device of FIGS. 1 to 5, the access devices of FIGS. 6 to 13 and FIGS. 16 to 18 and to the collection device of FIGS. 19 to 27 as well as to the collection devices of the apparatus of FIGS. 14 and 15, the vacuum applied to these devices, will depend on the use to which the devices are put, and the age of the subject, and will be selected by the surgeon to be of the appropriate value. The values of the vacuum described throughout the specification are to give a general indication of the level of vacuum which would be applied to the devices.

It will be readily apparent to those skilled in the art that any suitable type of filter which would be suitable for filtering out pathogens, bacteria, infections and/or other viruses, other particular matter and smoke from insufflating gas may be provided. It will also be appreciated that the minimum particle size to be filtered by the filter may be other than 0.05 microns. In some embodiments of the invention it is envisaged that the filter may be configured to filter out particles of minimum size greater than 0.05 microns, and in other embodiments of the invention the filter may be configured to filter out particles of minimum particle size less than 0.05 microns, and in some embodiments of the invention as low as 0.04 microns.

It will also be appreciated that the collection device, access devices, mouth and transanal entry devices, trocars and apparatus described herein as well as being adapted for collecting leaked insufflating gas which may contain pathogens and other particulate matter, will also collect insufflating gas containing smoke and other such matter and gases, and the insufflating gas containing smoke and other such matter, as well as pathogens and any other particular matter will be drawn by the vacuum system through the filter for filtering out any and all pathogens, particulate matter, smoke and other matter, so that clean filtered gases are safely dispersed into the environment without any detrimental or undesirable effects.

It is envisaged that in some embodiments of the access devices, for example, the mouth entry device of FIGS. 6 to 11, the trocar of FIGS. 12 and 13 and the transanal entry device of FIGS. 16 to 18 that in some embodiments of these access devices, the second inlet ports 69, in the case of the entry device of FIGS. 6 to 11 and the trocar of FIGS. 12 and 13 may be omitted, as may the second inlet ports 165 be omitted from the transanal entry device of FIGS. 16 to 18. In particular, in the case of the trocar of FIGS. 12 and 13 and the transanal entry device of FIGS. 16 to 18, if the provision of a vacuum on the second inlet ports 69 and 165 were to act to actively draw insufflating gas from the cavity being insufflated, the second inlet ports would be omitted or sealably closed.

It is also envisaged that in the case of the mouth and transanal entry devices, and the trocar, that the second inlet ports may be retained and the first inlet ports would be omitted or sealably closed.

It will of course be appreciated that while the collection device of FIGS. 1 to 5, the trocar of FIGS. 12 and 13 and the apparatus of FIGS. 14 and 15 have been described for collecting insufflating and other gases leaking from the peritoneal cavity, it will be appreciated that the collection device of FIGS. 1 to 5, the trocar of FIGS. 12 and 13 and the apparatus of FIGS. 14 and 15 may be used for collecting insufflating and other gases leaking from any other insufflated cavity in the body of a human or animal subject, and in which case, the collection device of FIGS. 1 to 5, the trocar of FIGS. 12 and 13 and the apparatus of FIGS. 14 and 15 will be located in an appropriate position in or on the subject. While the collection device of FIGS. 1 to 5 has been described as comprising a support element for supporting the patch in order to prevent the patch from collapsing under a vacuum applied to the collection chamber through the outlet port 25, it is envisaged that in some embodiments of the invention the support element may be omitted, and in which case, the material of the patch, and at least the material of the patch extending around the inner portion 23 of the patch and the outer peripheral portion 21 would be of a substantially self-supporting material, capable of supporting itself against the vacuum applied to the collection chamber. Such a material may be, for example, a rigid or a semi-rigid material.

While the collection chambers have been described as being located in the tubular members of the mouth and transanal entry devices adjacent the proximal flange thereof, in some embodiments of the invention it is envisaged that the collection chamber may be located in the proximal flange adjacent the tubular member. It is also envisaged that in the embodiment of the trocar that instead of the collection chamber being located in the tubular member of the trocar adjacent the housing thereof, the collection chamber may be located in the base plate of the housing adjacent the trocar.

It is also envisaged that while the second inlet ports of the access devices described with reference to FIGS. 6 to 13 and FIGS. 16 to 18 have been described as terminating in the instrument bore of the access devices adjacent the proximal end thereof, in some embodiments of the invention it is envisaged that the second inlet ports may terminate anywhere along the instrument bore between the proximal and distal end. Furthermore, it is envisaged that in some embodiments of the trocar, where a substantial length of the trocar may extend outwardly from the incision, the collection chamber may be located in the trocar spaced apart distally from the proximal end thereof, so that the first inlet ports would terminate in the outer surface of the trocar adjacent the incision.

It will be readily apparent to those skilled in the art that the collection devices and the access devices described herein, as well as the apparatus, may be connected to any suitable vacuum system, be it a fixed vacuum system installed in an operating theatre or a portable vacuum system dedicated for use with the collection device, the access device or the apparatus.

1-211. (canceled)

212. A collection device for minimising the dispersal of pathogens into the environment during insufflating of a lumen, vessel or cavity in a subject, the device comprising a patch terminating at an outer peripheral edge in a flexible peripheral membrane extending around and from the peripheral edge of the patch, a securing means for sealably securing the flexible peripheral membrane to a subject spaced apart around an opening through which an access device enters the subject, the patch having an access opening extending therethrough spaced apart inwardly from the outer peripheral edge thereof for accommodating the access device therethrough to the opening in the subject, the patch being adapted so that when secured to the subject by the securing means with the access device passing through the access opening in the patch and passing through the opening in the subject, the patch defines with the subject and the access device a collection chamber for collecting insufflating gas leaking through the opening in the subject around the external surface of the access device, and an outlet port is provided extending from the patch communicating with the collection chamber, the outlet port being adapted for coupling to a vacuum generating system for drawing gas from the collection chamber.

213. A collection device as claimed in claim 212 in which the patch is adapted to define an annular collection chamber extending around the access device when secured to the subject by the securing means, and preferably, the patch comprises an inner portion adjacent and extending around the access opening adapted to tightly engage the access device, and advantageously, the inner portion of the patch adjacent and extending around the access opening is adapted to sealably engage the access device, and preferably, the inner portion of the patch adjacent and extending around the access opening comprises a resilient material, and advantageously, the inner portion of the patch adjacent and extending around the access opening comprises a flexible material, and preferably, the inner portion of the patch adjacent and extending around the access opening comprises an elastic material.

214. A collection device as claimed in claim 212 in which the patch comprises an intermediate portion located between the peripheral membrane and the inner portion of the patch extending around the inner portion, the intermediate portion of the patch comprising a flexible material, and preferably, at least the intermediate portion of the patch comprises a semi-rigid material, and preferably, at least the intermediate portion of the patch comprises a substantially self-supporting material, and advantageously, the intermediate portion of the patch comprises a non-self-supporting material, and preferably, the patch comprises a material impermeable to insufflating gases, and advantageously, the patch is integrally formed from a single sheet of material.

215. A collection device as claimed in claim 212 in which a support element is provided for supporting the patch to define with the access device and the subject the collection chamber, and preferably, the support element is adapted for locating between the patch and the subject, and advantageously, the support element is adapted to extend around the patch spaced apart from the access opening, and preferably, the support element comprises a support framework, and preferably, the support framework comprises a lower peripheral frame for locating adjacent the subject, and an upper peripheral frame spaced apart above the lower peripheral frame, the upper peripheral frame being joined to the lower peripheral frame by at least one strut extending from the upper peripheral frame to the lower peripheral frame, and advantageously, the upper peripheral frame extends around but spaced apart outwardly from the access opening, and preferably, the upper peripheral frame is spaced apart outwardly from the access opening to permit flexing of the inner portion of the patch, and advantageously, the lower peripheral frame extends around adjacent but spaced apart inwardly from the outer peripheral edge of the patch, and preferably, the upper peripheral frame comprises an upper circular ring member, and advantageously, the lower peripheral frame comprises a lower circular ring member, and preferably, the diameter of the lower peripheral frame is greater than the diameter of the upper peripheral frame, and advantageously, the securing means comprises an adhesive coated onto the peripheral membrane, and preferably, the peripheral edge in which the patch terminates defines a circle, and advantageously, the access opening is of circular shape, and preferably, the access opening is adapted to accommodate a trocar therethrough, and advantageously, the access opening is adapted to accommodate an endoscope, and preferably, the access opening is adapted to accommodate a colonoscope, and advantageously, a filter is provided for filtering gases drawn from the collection chamber, and preferably, the filter is adapted for coupling to the outlet port, and advantageously, the filter is adapted to filter out one or more of pathogens, bacteria, infections and viruses from the gases passing therethrough, and preferably, the filter is adapted to filter out particles of size greater than 0.1 microns entrained in the gases passing therethrough, and advantageously, the filter is adapted to filter out particles of size greater than 0.05 microns entrained in the gases passing therethrough, and preferably, the filter is adapted to filter out particles of size greater than 0.04 microns entrained in the gases passing therethrough.

216. A collection device as claimed in claim 212 further comprising an access device for accommodating an instrument to a lumen, vessel or cavity in a subject, the access device extending through the access opening in the patch and sealably engaged in the access opening, and defining with the patch an annular collection chamber, and preferably, the access device comprises a trocar.

217. A method for minimising dispersal of pathogens into the environment during insufflating of a lumen, vessel or cavity of a subject, the method comprising:
providing a patch terminating in an outer peripheral edge and having an access opening extending therethrough spaced apart inwardly from the peripheral edge thereof,
placing the patch topically on an area of the skin of the subject containing an opening through which an access device enters the subject,
sealably securing the patch to the skin of the subject adjacent the peripheral edge thereof spaced apart from and extending around the opening in the subject, entering the access instrument through the access opening to the opening in the subject with a portion of the patch adjacent the access opening sealably engaging the access device with the patch defining with the access device and the skin of the subject a collection chamber for collecting insufflating gas leaking from the opening in the subject along the outer surface of the access device, and
applying a vacuum to an outlet port from the patch to draw the insufflating gases from the collection chamber.

218. A method as claimed in claim 217 in which the patch is sealably secured to the skin of the subject with the patch defining with the access device and the skin of the subject the collection chamber in the form of an annular collection chamber, and preferably, the patch comprises an inner portion adjacent and extending around the access opening adapted to tightly engage the access device, and advantageously, the inner portion of the patch adjacent and extending around the access opening comprises a resilient material, and preferably, the inner portion of the patch adjacent and extending around the access opening comprises a flexible material, and advantageously, the inner portion of the patch adjacent and extending around the access opening comprises an elastic material.

219. A method as claimed in claim 217 in which the patch comprises an intermediate portion located between the flexible peripheral membrane and the inner portion of the patch and extending around the inner portion, the intermediate portion of the patch comprising a flexible material, and preferably, the intermediate portion of the patch comprises a semi-rigid material, and advantageously, the intermediate portion of the patch comprises a substantially self-supporting material, and preferably, the intermediate portion of the patch comprises a non-self-supporting material.

220. A method as claimed in claim 217 in which the patch is supported by a support element to define the collection chamber, and preferably, the support element is located between the patch and the skin of the subject, and advantageously, a support element is located around the patch spaced apart from the access opening, and preferably, the support element comprises a support framework, and advantageously, the patch is integrally formed from a single sheet of material, and preferably, the patch comprises a material impermeable to an insufflating gases, and advantageously, the patch is secured to the skin of the subject by an adhesive, and preferably, the access opening in the patch is adapted to accommodate a trocar therethrough, and advantageously, the gases drawn from the collection chamber are filtered through a filter adapted to filter out one or more of pathogens, bacteria, infections and viruses from the gases passing therethrough, and preferably, the filter is adapted to filter out particles of size greater than 0.1 microns entrained in the gases passing therethrough.

221. An access device for accommodating an instrument through an opening in a human or animal subject, the access device being adapted to extend through the opening in the subject and having an instrument bore extending therethrough for accommodating the instrument therethrough, the access device having an inner surface defining the instrument bore, and an external outer surface, wherein the access device comprises a collection chamber located in the access device between and spaced apart from the inner and outer surfaces thereof and extending at least partly around the instrument bore, an outlet port communicating with the collection chamber and adapted for connecting to a vacuum system, and at least one first inlet port communicating with and extending from the collection chamber and terminating in one of an inner surface and an outer surface of the access device, the at least one first inlet port being adapted to draw gases into the collection chamber in response to a vacuum being applied to the outlet port.

222. An access device as claimed in claim 221 in which the collection chamber comprises an annular chamber extending around the instrument bore, and preferably, the collection chamber extends completely around the instrument bore, and advantageously, the at least one first inlet port terminates in the one of the inner surface and the outer surface of the access device towards a proximal end thereof, and preferably, the at least one first inlet port terminates in the one of the inner surface and the outer surface of the access device adjacent the proximal end thereof, and advantageously, the at least one first inlet port terminates in the outer surface of the access device for drawing gases leaking through the opening in the subject past the outer surface of the access device, and preferably, a plurality of the first inlet ports extend from and communicate with the collection chamber and terminate in the outer surface of the access device spaced apart circumferentially around the access device, and advantageously, the first inlet ports are equi-spaced apart circumferentially around the access device.

223. An access device as claimed in claim 221 in which at least one second inlet port communicates with and extends from the collection chamber and terminates in the inner surface of the access device, the at least one second inlet port being adapted to draw gases into the collection chamber in response to a vacuum being applied to the outlet port, and preferably, the at least one second inlet port communicates with the instrument bore of the access device, and advantageously, the at least one second inlet port terminates in the inner surface of the access device towards the proximal end thereof, and preferably, the at least one second inlet port terminates in the inner surface of the access device adjacent the proximal end thereof, and advantageously, a plurality of the second inlet ports extend from and communicate with the collection chamber and terminate in the inner surface of the access device spaced apart circumferentially around the inner surface, and preferably, the second inlet ports are equi-spaced apart circumferentially around the inner surface of the access device, and advantageously, the collection chamber is located towards the proximal end of the access device, and preferably, the collection chamber is located adjacent the proximal end of the access device.

224. An access device as claimed in claim 221 in which the access device terminates in one of a proximal flange and a proximal housing, the one of the proximal flange and the proximal housing defining a part of the outer surface of the access device.

225. An access device as claimed in claim 224 in which the at least one first inlet port terminates in the outer surface of the access device adjacent but spaced apart distally from the proximal housing, and preferably, the at least one first inlet port terminates in the part of the outer surface of the access device defined by the proximal housing, and advantageously, the at least one first inlet port terminates in the part of the outer surface of the access device defined by the proximal housing towards the access device, and preferably, the at least one first inlet port terminates in the part of the outer surface of the access device defined by the proximal housing adjacent but spaced apart from the access device, and advantageously, the at least one first inlet port terminates in the outer surface of the access device intermediate the access device and the proximal housing, and preferably, the at least one first inlet port terminates in the part of the outer surface of the access device defined by the proximal housing spaced apart from the access device.

226. An access device as claimed in claim 224 in which the at least one first inlet port terminates in the outer surface of the access device adjacent but spaced apart distally from the proximal flange, and preferably, the at least one first inlet port terminates in the part of the outer surface of the access device defined by the proximal flange, and advantageously, the at least one first inlet port terminates in the part of the outer surface of the access device defined by the proximal flange towards the access device, and preferably, the at least one first inlet port terminates in the part of the outer surface of the access device defined by the proximal flange adjacent but spaced apart from the access device, and advantageously, the at least one first inlet port terminates in the outer surface of the access device intermediate the access device and the proximal flange, and preferably, the at least one first inlet port terminates in the part of the outer surface of the access device defined by the proximal flange spaced apart from the access device, and advantageously, the outlet port extends from the collection chamber and terminates in the part of the outer surface of the access device formed by the one of the proximal flange and the proximal housing, and preferably, the outlet port extends from the collection chamber and terminates in or extends through the outer surface of the access device, and advantageously, a filter is provided for filtering out one or more of pathogens, bacteria, infections and viruses from gases drawn through the outlet port, and preferably, the filter is adapted for coupling to the outlet port.

227. An access device as claimed in claim 221 in which the access device comprises one of a trocar, a mouth entry device for accommodating an instrument orally into the subject, and a transanal entry device for accommodating an instrument anally into the subject.

228. A method for minimising dispersal of gases escaping from an opening in a subject through which an access device is located for accommodating an instrument into the subject, the method comprising providing the access device with an instrument bore extending therethrough for accommodating the instrument therethrough and providing the access device with an inner surface defining the instrument bore therethrough and an external outer surface, providing a collection chamber located in the access device between and spaced apart the inner and outer surfaces thereof and extending at least partly around the instrument bore providing at least one outlet port communicating with the collection chamber adapted to communicate with a vacuum system, providing at least one first inlet port communicating with and extending from the collection chamber and terminating in one of the inner surface and the outer surface of the access device, locating the access device in the opening in the subject, and applying a vacuum to the outlet port for drawing gases escaping from the subject into the collection chamber through the at least one first inlet port.

229. A method as claimed in claim 228 in which the collection chamber comprises an annular chamber extending around the instrument bore, and preferably, the collection chamber extends completely around the instrument bore, and advantageously, the at least one first inlet port terminates in the one of the inner surface and the outer surface of the access device towards a proximal end thereof, and preferably, the at least one first inlet port terminates in the outer surface of the access device for drawing gases leaking through the opening in the subject past the outer surface of the access device, and advantageously, a plurality of the first inlet ports extend from and communicate with the collection chamber and terminate in the outer surface spaced apart circumferentially around the access device, and preferably, the first inlet ports are equi-spaced apart circumferentially around the access device.

230. A method as claimed in claim 228 in which at least one second inlet port communicates with and extends from the collection chamber and terminates in the inner surface of the access device, the at least one second inlet port being adapted to draw gases into the collection chamber in response to a vacuum being applied to the outlet port, and preferably, the at least one second inlet port communicates with the instrument bore, and advantageously, the at least one second inlet port is provided towards the proximal end of the access device, and preferably, a plurality of the second inlet ports communicate with and extend from the collection chamber, the second inlet ports being spaced apart circumferentially around the inner surface of the access device, and advantageously, the second inlet ports are equi-spaced apart circumferentially around the inner surface of the access device, and preferably, the outlet port extending from the collection chamber terminates in or extends through the outer surface of the access device, and advantageously, the collection chamber is located adjacent the proximal end of the access device, and preferably, gases drawn through the outlet port are filtered by a filter, and advantageously, one or more of pathogens, bacteria, infections and viruses are filtered out from gases drawn through the outlet port.

231. A method as claimed in claim 228 in which the access device comprises one of:
a trocar, and the trocar is located in the opening formed by an incision formed in the subject with the at least one first inlet port located externally of the incision but adjacent thereto,
a mouth entry device for accommodating an instrument orally into the subject, and the mouth entry device is located in the mouth of the subject between the upper and lower teeth of the subject with the at least one first inlet port located in or externally of the mouth of the subject but adjacent the mouth of the subject, and
a transanal entry device for accommodating an instrument anally into the subject, and the transanal entry device is located in the anus of the subject, with the at least one first inlet port located externally of the anus but adjacent thereto.
